(19) United States
(12) Patent Application Publication
(10) Pub. No.: US 2009/0183739 A1
(43) Pub. Date: Jul. 23, 2009

(54) METHODS AND DEVICES FOR IMPROVING EFFICACY OF NON-INVASIVE VENTILATION

(75) Inventor: Anthony D. Wondka, Thousand Oaks, CA (US)

Correspondence Address: PATTON BOGGS LLP, 8484 WESTPARK DRIVE, SUITE 900, MCLEAN, VA, 22102, US

(73) Assignee: MenloLife, Inc.

(21) Appl. No.: 12/355,753

(22) Filed: Jan. 16, 2009

Related U.S. Application Data
(60) Provisional application No. 61/006,548, filed on Jan. 18, 2008.
(60) Provisional application No. 61/106,414, filed on Oct. 17, 2008.

(51) Int. Cl. A61M 16/06

(52) U.S. Cl. 128/207.18

(57) ABSTRACT

A nasal ventilation interface including a manifold a nasal cushions. The manifold is configured with compound arcuate curves for optimizing fit and performance. The ventilation gas supply hose is attached to only one side of the manifold at any given time, thereby freeing up the opposite side of the user's face to enhance comfort and tolerance while sleeping.

### CROSS REFERENCE TO RELATED APPLICATIONS

This application claims priority to U.S. Provisional Patent Application Ser. No. 61/006,548 filed Jan. 18, 2008, and to U.S. Provisional Patent Application Ser. No. 61/106,414 filed Oct. 17, 2008, each of which is incorporated herein by reference in its entirety. This application also incorporates by reference in their entireties the following: U.S. patent application Ser. No. 12/076,062, filed Mar. 13, 2008; U.S. Pat. No. 7,406,966, issued Aug. 5, 2008; U.S. Provisional Patent Application Ser. No. 60/495,812 filed Aug. 18, 2003; and U.S. Provisional Patent Application Ser. No. 60/511,820, filed Oct. 14, 2003.

### FIELD OF INVENTION

This invention relates to non-invasive ventilation (NIV) patient interface devices which provide a route of air entry into a patient's airway and lung. More particularly, this invention can be applied to Obstructive Sleep Apnea (OSA), a condition where the upper airway obstructs; however, the teachings herein are applicable to other respiratory conditions.

### BACKGROUND OF THE INVENTION

NIV patient interface devices are fastened to the outside of a patient's nose and/or mouth and do not penetrate deep into the airway. These devices are used in a variety of medical procedures, such as emergency ventilation, anesthesia delivery and recovery, aerosolized medication delivery, augmentation of natural breathing, supplemental oxygen delivery, mechanical ventilation, weaning from mechanical ventilation and for treating OSA. In the case of OSA, continuous positive airway pressure (CPAP) or continuous variable-level positive airway pressure (VPAP) is delivered through the interface device into the patient's airway during sleep to prevent airway obstruction. OSA is unique to all positive airway pressure (PAP) applications in that the therapy is preferably minimally obtrusive in order to not disrupt the patient while sleeping, whereas in other PAP applications disrupting sleep is of little concern. There are three different forms of NIV interface devices; Nasal Interfaces, Oral Interfaces and combined Oral-Nasal Interfaces. Of the Nasal Interface form, there are two categories: Over the nose Nasal Masks and Nasal Cannulae or Nasal Pillows that impinge on the nostrils.

### SUMMARY OF THE INVENTION

The present invention provides a PAP nasal pillows ventilation interface device preferably comprising or providing at least one of the following features: a manifold having arcuate curves that preferably anatomically conforms to the user's face, a gas supply tubing assembly that attaches to only one side of the manifold at any given time and extends to only one side of the face at any given time, a low flow resistance design, a comfortable and effective seal against the nostrils without deep penetration into the nostril, a design that is comfortable and unobtrusive to the overall face, a device easy to attach and remove, and a device that allows the user to lay on his or her side without discomfort or device malfunction. The manifold and gas supply preferably may be designed with curves to match the user's anatomy and position to the device in the most comfortable parts of the face. In particularly preferred embodiments, the combination of a low profile design, a unilateral attachment of the gas supply, a rotational connection, and curves and shape of the manifold, preferably allow ventilation interface devices according to certain embodiments of the invention to be minimally obtrusive and comfortable to the user. Additionally, the manifold and gas supply preferably may be worn by the patient using a connector between the head strap and manifold that preferably includes at least two different adjustments, thus preferably facilitating fit to individual head shapes and sizes and maximum comfort.

In certain embodiments, the present invention preferably provides an OSA PAP nasal interface that includes, provides, or facilitates at least one of the following: (1) low resistance and low noise gas flow dynamics; (2) a comfortable and effective nostril seal without requiring deep penetration into the nose; (3) a simultaneously comfortable, unobtrusive and non-irritating system to retain the device to the nose, face and head; (4) a system or device that is easy to attach and remove; and (5) an the overall apparatus that is minimally obtrusive, comfortable and ergonomic, allowing a user to speak, see, wear glasses, drink, and talk on the phone while being worn before falling asleep, and allowing the user to shift sleep positions and comfortably lay on their side during sleep without shifting the device or dislodging the portion that seals to the nose.

In certain embodiments, the invention provides a nasal interface for providing ventilation gas to assist in inflating a lung of a person, comprising: (a) a tubular manifold adapted to be positioned inferiorly to the nostrils and superior to the mouth comprising: (1) an axially tubular structure comprising compound arcuate curves curving bilaterally from a manifold midline in a lateral and posterior direction, (2) a left lateral end and a right lateral end, (3) at least two gas flow openings on the superior side of the manifold, and (4) and a closure connected to the one lateral end; (b) a pair of tubular nasal cushions each comprising a proximal end base attached to one of the at least two gas flow openings, and each extending to a distal end adapted to impinge with and seal against the nostrils; (c) a ventilation gas supply assembly comprising a distal end adapted to connect to the lateral end of the manifold opposite the lateral end connected to the closure, the connection adapted to rotate in at least one plane, and a proximal end adapted to attach to a ventilator; (d) a head strap assembly; and (e) a connector connecting the head strap assembly to the manifold, the connector comprising two movable connections, wherein a first movable connection between the manifold and connector allows rotation of the manifold cross section in the sagittal plane, and a second movable connection between the head strap and connector allows pivoting of the head strap in the inferior-posterior direction.

The compound arcuate curves of the manifold may be shaped to match the anatomy of a user's face between the nose and mouth and to the sides of the nose and wherein the curves are stabilized to maintain substantial contact of the skin side of the manifold with the user's anatomy. The compound arcuate curves of the manifold may include a concave curve on the superior side of the manifold in the coronal plane curving or angling superiorly from the midline, wherein the concave curve comprises a substantially V-shaped or U-shaped curve symmetrical about the midline, and wherein the substantially V-shaped or U-shaped curve comprises a 110-170 degree included angle. The compound arcuate curves of the manifold may include a concave curve on the superior side of the manifold in the coronal plane curving or angling superiorly from the midline and a concave curve on the inferior side of the manifold in the coronal plane curving or angling inferiorly from the midline. The compound arcuate curves of the manifold may further comprise a taper, wherein the taper transitions from a first larger cross sectional dimension at a medial location to a second smaller cross sectional dimension at a lateral location.

The manifold may further comprise at least one exhalation flow vent port in a location selected from the group consisting of: at least one channel in the wall of the manifold at a inferior-anterior location opposite to and substantially aligned with the gas flow openings, at least one channel in the manifold lateral end closure, at least one channel in the ventilation gas supply assembly substantially aligned with the direction of exhaled gas flow. The cross sectional profile of at least one section of the manifold may be shorter in a first axis and longer in a second axis orthogonal to the first axis, wherein the second axis is positioned parallel to the face. The manifold may further comprise a middle section in the location of the gas flow openings, and the middle section may comprise (i) a cross sectional superior surface in the sagittal plane that angles inferiorly 5-30 degrees from the anterior side to the posterior side and (ii) a cross sectional posterior surface in the sagittal plane that angles posteriorly 5-30 degrees from the superior side to the inferior side. The manifold may comprise a material having Shore 10-30A hardness, and may further comprise at least one stiffening member, wherein the stiffening member is selected from the group consisting of: a strip of semi-rigid plastic, a strip of metal alloy, a radial rib, and an axial rib. The manifold may comprise a material having Shore 10-30A hardness, and may further comprise at least one stiffening member, wherein the stiffening member stabilizes and reduces the compressibility of the tubular manifold structure. The manifold may comprise a material having a Shore 10-30A hardness, and may further comprise at least one malleable member adapted to allow a user to bend and slightly reshape the curvature of the manifold to fit the user's individual anatomy. The superior surface of the manifold may comprise a convolution in the wall around the gas flow openings.

The nasal interface assembly may further comprise a rigid cylindrical sleeve inside the manifold at each lateral end. The manifold may further comprise a groove in the wall near each of the two lateral ends, wherein groove may be adapted for attachment of the head strap assembly connector. The manifold may further comprise a spacing adjuster adapted to adjust the distance between the nasal sealing cushions, wherein the spacing adjuster may be selected from the group consisting of: a self sealing slot, an adjustable ring, and a replaceable adaptor. The manifold may comprise separate left and right tubular sections, wherein the two tubular sections may be connected by a tubular interconnecting member.

The nasal cushions may further comprise a round proximal base and an oval distal end, and the nasal cushion proximal base and manifold superior side gas flow openings may comprise a mating rotatable connection adapted to rotate the nasal cushion about its attachment to the manifold; and the nasal sealing cushions may further comprise: (1) a convolution in the wall near the proximal end base, wherein the convolution is adapted to enable angular flexing or axial compression of the nasal cushion, (2) construction using a material having a durometer of 10 to 50 Shore A, the material selected from the category of: a thermoplastic, an elastomer, or a thermoplastic elastomer, (3) an enlarged effective diameter at a distance 2-10 mm from proximal to its distal end wherein the enlarged effective diameter creates the general configuration of a step, and wherein the enlarged effective diameter is a dimension larger than the nostril rim inner diameter, and is in the range of 7 mm to 20 mm and wherein the step engages the nostril rim to effect a seal and to prevent over penetration of the nasal sealing cushion into the nostril, and (4) a second seal at the distal tip of the cushion which is adapted to seal inside the nostril, the distal tip seal comprising a feature selected from the group consisting of a flare, a ring, an effective diameter larger than the nostril dimension, and an inflatable wall. The nasal cushions may further comprise an inflatable outer wall sealing surface adapted to inflate by the flow of ventilation gas flowing to the patient, wherein the inflation outwardly expands the nasal cushion outer wall sealing surface to press against the nostril foramen.

The manifold superior side may comprise a raised tubular extension for attachment of nasal cushions, and the nasal cushions may be (a) adapted to slip over the raised tubular extension, and (b) comprised of a viscoelastic shape memory material selected from the group consisting of: an elastomer, a hydrogel, and a foam, wherein the material possess a recovery of greater than 80% in 3 seconds from 50% compression.

The nasal interface assembly may further comprise a pad attached to the posterior side of manifold, wherein the pad may extend to the lateral sides of the nose, and the pad may further comprise a connector at the lateral ends to connect to the head strap assembly.

The connector connecting the head strap assembly to the manifold may further comprise: (1) a left and right connector adapted to be removably and rotatably attachable to the manifold, with at least two rotational attachment positions, and (2) a left and right connecting plate connected to the left and right connector at the posterior side of the manifold; and the plates may comprise (a) a pad on the posterior skin side of the plate and (b) a connection joint for connecting to the head strap assembly, and the head strap assembly may further comprise a forward left and right strap comprising: a soft material on the skin size of the strap, and a semi-rigid strip, wherein the semi-rigid strip comprises an attachment means to attach to the attachment plate connection joint, and wherein the attachment means comprises a pivoting connection between the forward strap and the attachment plate adapted to provide multiple rotational positions of the strap relative to the plate. The head strap assembly may further comprise a malleable member adapted to shape the assembly and resist deformation of a desired shape.

The distal end of the ventilation gas supply assembly may further comprise: an elbow connector assembly adapted to connect to one side of the manifold wherein the elbow connector assembly comprises at least one rotational connector adapted to rotate in at least two planes, wherein the rotation allows positioning of the gas supply hose to multiple positions. The manifold and ventilation gas supply assembly may be further adapted to removably and switch-ably attach the ventilation gas supply to either the left or right lateral end of the manifold. The axially tubular structure may further comprise an axial arc length longer than the width of the base of the nose of the user. The distal ends of the nasal cushions may have an outer dimension of 7 mm to 17 mm.

In certain embodiments, the invention provides a kit comprising a nasal interface assembly comprising: (a) a tubular manifold adapted to be positioned inferiorly to the nostrils and superior to the mouth comprising: (1) an axially tubular structure comprising compound arcuate curves curving bilaterally from a manifold midline in a lateral and posterior direction, (2) a left lateral end and a right lateral end, (3) at least two gas flow openings on the superior side of the manifold, and (4) and a closure connected to the one lateral end; (b) a pair of tubular nasal cushions each comprising a proximal end base attached to one of the at least two gas flow openings, and each extending to a distal end adapted to impinge with and seal against the nostrils; (c) a ventilation gas supply assembly comprising a distal end adapted to connect to the lateral end of the manifold opposite the lateral end connected to the closure, the connection adapted to rotate in at least one plane, and a proximal end adapted to attach to a ventilator; (d) a head strap assembly; and (e) a connector connecting the head strap assembly to the manifold, the connector comprising two movable connections, wherein a first movable connection between the manifold and connector allows rotation of the manifold cross section in the sagittal plane, and a second movable connection between the head strap and connector allows pivoting of the head strap in the inferior-posterior direction.

The kit may further comprise (a) at least one manifold of a first size and a second manifold of a second size, (b) at least one head strap assembly of a first size and a second head strap assembly of a second size, and (c) at least one pair of nasal sealing cushions of a first size and a second pair of nasal sealing cushions of a second size. The distal ends of the nasal cushions may have an outer dimension of 7 mm to 17 mm.

In certain embodiments, the invention provides a method for supplying ventilation gas to a person to assist in inflating the lung of the person using a nasal interface assembly, the method comprising: (a) placing a compound arcuately curved tubular manifold between the user's nose and mouth and stabilizing it against the skin; (b) removably attaching a pair of tubular nasal cushions to the superior side of the manifold, wherein the nasal cushions to impinge on the nostril rims, and limiting penetration of the cushions into the nostrils by including an enlarged step on each cushion; (c) connecting a distal end of a ventilation gas supply assembly to a first lateral end of the manifold to create a rotatable connection in at least one plane, and connecting the proximal end of the ventilation gas supply hose to a ventilator; (d) sealing the second lateral end of the manifold opposite the connection of the ventilation gas supply hose assembly; and (e) fastening the manifold to the user's face by attaching a head strap assembly to the manifold with at least one adjustable attachment between the manifold and head strap assembly.

The method may further comprise: switching the ventilation gas supply assembly attachment to the second lateral end of the manifold and switching the sealing of the second lateral end of the manifold to the first lateral end of the manifold. The method may further comprise moving the position of the ventilation gas supply hose from one position on the face to a second position on the face using rotatable connections. The method may further comprise securing the user interface to the patient's face by pulling the manifold in a posterior and superior direction using the head strap assembly coupled to the lateral ends of the manifold and extending straps of the head strap assembly to the back and top of the head, and the manifold may be rotationally adjusted by the coupling, and the straps may be pivoted by the coupling.

The method may further comprise inflating the nasal sealing cushions with gas supplied from the ventilation gas supply. The method may further comprise adjusting the nasal cushions, the adjustment selected from the group of: adjusting the included angle between the nasal sealing cushions, adjusting the space between the nasal sealing cushions, rotating the nasal cushions at the connection between the nasal cushions and the manifold.

The method may further comprise exhausting exhalation flow, the exhausting selected from the group consisting of: exhausting through ports positioned in wall of the manifold opposite to the nasal cushions, exhausting through the lateral end closure, and exhausting through ports in the ventilation gas supply. The method may further comprise delivering a supplemental flow of oxygen gas to the patient by connecting a supply of oxygen gas to the manifold.

In certain embodiments, the invention provides a nasal interface assembly comprising: (a) a tubular manifold adapted to be positioned inferiorly to the nostrils and superior to the mouth, comprising: (1) an axial length longer than the base of the nose and a left and right lateral end, (2) a centerline arc axis comprising compound arcuate curves with a first section curving bilaterally from the midline comprising a posterior and superior curve, two second sections attached to the left and right lateral end of the first section and curving laterally and posteriorly to the left and right lateral ends of the manifold, (3) at least two gas flow openings on the superior surface of the manifold, (4) a connector on each of the left and right lateral ends of the manifold adapted to attach a gas supply assembly to one end and a closure to the other end, (5) at least one connector on the superior surface adapted to attach two nasal sealing cushions to communicate with the gas flow openings, (6) a connector on each of the left and right lateral ends for attaching a head strap assembly, (7) exhalation exhaust ports; (b) a pair of tubular nasal cushions comprising a proximal end base adapted to removably attach to a connector on the superior side of the manifold and to be in communication with the gas flow openings, and comprising a distal end adapted to impinge with and seal against the nostrils; (c) a ventilation gas supply assembly comprising: (1) a distal end elbow connector assembly adapted to attach to one lateral side of the manifold and adapted to rotate in multiple planes, (2) a flex hose in communication with the distal elbow assembly, and (3) a proximal end connection adapted to attach to a ventilator; (d) a closure adapted to attach to a lateral side of the manifold; and (e) a head strap assembly comprising: (1) a left and right connector adapted to rotatably attach to the attachment means on the manifold, (2) a left and right attachment plate attached to the left and right connector wherein the left and right attachment plates are adapted with curves to stabilize the plates in contact with the skin, (3) a left and right strap movably connected to the left and right attachment plate and extending posteriorly to above the ears and joining at the rear of the head.

Additional features, advantages, and embodiments of the invention may be set forth or are apparent from consideration of the following detailed description, drawings, and claims. Moreover, it is to be understood that both the foregoing summary of the invention and the following detailed description are exemplary and intended to provide further explanation without limiting the scope of the invention as claimed.

### BRIEF DESCRIPTION OF THE FIGURES

The accompanying drawings, which are included to provide a further understanding of the invention and are incorporated in and constitute a part of this specification, illustrate preferred embodiments of the invention and together with the detailed description serve to explain the principles of the invention. In the drawings:

FIG. 1 illustrates an isometric view of an embodiment of the invention when placed on a person's head.

FIG. 2 illustrates a front view of an embodiment of the invention, showing a manifold, head strap assembly, and gas supply attached to one side of the manifold in accordance with an embodiment of the present invention.

FIG. 3 illustrates a top view of an embodiment of the invention.

FIG. 4 illustrates a right side view of an embodiment of the invention when placed on a person's head.

FIG. 5 illustrates a top view of an embodiment of a manifold in accordance with an embodiment of the present invention.

FIG. 6 illustrates a front view of an embodiment of a manifold in accordance with an embodiment of the present invention.

FIG. 7 illustrates a front view of an embodiment of a manifold in accordance with an embodiment of the present invention.

FIG. 8A illustrates a front view of an embodiment of a manifold in accordance with an embodiment of the present invention.

FIG. 8B illustrates a front view of an embodiment of a manifold in accordance with an embodiment of the present invention.

FIG. 8C illustrates a front view of an embodiment of a manifold in accordance with an embodiment of the present invention.

FIG. 9A illustrates a front view of an embodiment of a manifold in accordance with an embodiment of the present invention.

FIG. 9B illustrates a cross section of a non-round mid section of the manifold of FIG. 9A, with a flattened superior surface.

FIG. 9C illustrates a cross section of a round profile of a lateral section of the manifold of FIG. 9A.

FIG. 10A illustrates a front view of an embodiment of a manifold in accordance with an embodiment of the present invention.

FIG. 10B illustrates a cross section of a medial section of the manifold of FIG. 10A.

FIG. 10C illustrates a cross section near the cross section of FIG. 10B.

FIG. 10D illustrates a longitudinal cross section of the manifold of FIG. 10A.

FIG. 11 illustrates an alternate cross section of a medial section of the manifold of FIG. 10A.

FIG. 12A illustrates a front view of an embodiment of a manifold in accordance with an embodiment of the present invention.

FIG. 12B illustrates a cross sectional view through a mid section of the manifold of FIG. 12A.

FIG. 12C illustrates a cross sectional view though a lateral section of the manifold of FIG. 12A.

FIG. 13 illustrates a top view of an embodiment of a manifold in accordance with an embodiment of the present invention.

FIG. 14A illustrates a top view of an embodiment of a manifold in accordance with an embodiment of the present invention.

FIG. 14B illustrates a top view of an embodiment of a manifold in accordance with an embodiment of the present invention.

FIG. 14C illustrates a cross sectional view of the manifold of FIG. 14B.

FIG. 14D illustrates a top view of a n embodiment of a manifold in accordance with an embodiment of the present invention.

FIG. 15A illustrates a front view of an embodiment of a manifold in accordance with an embodiment of the present invention.

FIG. 15B illustrates a cross sectional view of a boss of FIG. 15A with a nasal cushion attached.

FIG. 15C illustrates a cross sectional view of the assembly of FIG. 15B while in use impinging on the user's nostril.

FIG. 16A illustrates a side view of an embodiment of a nasal cushion in accordance with an embodiment of the present invention.

FIG. 16B illustrates a top view of the nasal cushion of FIG. 16A.

FIG. 16C illustrates a front view of an embodiment of a nasal cushion in accordance with an embodiment of the present invention.

FIG. 16D illustrates a front view of an embodiment of a nasal cushion in accordance with an embodiment of the present invention.

FIG. 17A illustrates front view of an embodiment of a nasal cushion in accordance with an embodiment of the present invention.

FIG. 17B illustrates a front view of the nasal cushion of 17A with a sealing wall inflated.

FIG. 17C illustrates a cross sectional view of the nasal cushion of 17B.

FIG. 17D illustrates a detailed view of a portion of FIG. 17C.

FIG. 18A illustrates a front view of an embodiment of a nasal cushion assembly in accordance with an embodiment of the present invention.

FIG. 18B illustrates a front view of an embodiment of a nasal cushion assembly in accordance with an embodiment of the present invention.

FIG. 19 illustrates a front or side view of an embodiment of a swivel elbow connector assembly between a gas supply hose and a manifold, in accordance with an embodiment of the present invention.

FIG. 20 illustrates an isometric view of a right connecting assembly for connecting a head strap assembly to a manifold, in accordance with an embodiment of the present invention.

FIG. 21 illustrates an isometric view of an embodiment of a head strap assembly in accordance with an embodiment of the present invention.

FIG. 22 illustrates a side view of the right side of an embodiment of a head strap assembly in accordance with an embodiment of the present invention.

FIG. 23A illustrates a front view of an embodiment of a manifold end cap in accordance with an embodiment of the present invention.

FIG. 23B illustrates an end view of the end cap of FIG. 23A.

FIG. 23C illustrates a cross sectional view of the end cap of FIG. 23B, illustrating a ramp.

FIG. 24A illustrates a front view of an embodiment of a manifold assembly, in accordance with an embodiment of the present invention.

FIG. 24B illustrates a cross section of FIG. 24A at the line A-A.

FIG. 25 illustrates a top view of an embodiment of an interface manifold assembly, in accordance with an embodiment of the present invention.

FIG. 26 illustrates a top view of an embodiment of a manifold, in accordance with an embodiment of the present invention.

### DETAILED DESCRIPTION OF THE PREFERRED EMBODIMENTS

The present invention provides a nasal pillows NIV device. In certain embodiments, a manifold, which may also be referred to as a “base manifold”, preferably may be positioned under the nose. In certain preferable embodiments, the manifold may be an low profile and may anatomical conform to, or match, the user's face. The ventilation gas supply preferably may be unilaterally attached to only one side of the manifold at any given time and routed to only one side of the face at any given time to preferably provide a high degree of comfort and un-obtrusiveness for some users especially when the user sleeps on his or her side, rather than being attached bilaterally to both sides of the manifold. Moreover, the ventilation gas supply preferably may be connected with a rotational connection that can rotate in at least one plane. The manifold and gas supply preferably may be designed with curves to preferably match the user's anatomy and position to the device on the most comfortable parts of the face. The combination of the low profile design, the unilateral attachment of the gas supply, the rotational connection, and the curves and shape, preferably allow this design to be minimally obtrusive and comfortable to the user. Additionally, the manifold and gas supply preferably may be worn by the patient using a connector between the head strap and manifold that preferably includes at least two different adjustments, thus facilitating fit to individual head shapes and sizes and maximum comfort.

FIG. 1 illustrates an isometric view of an embodiment of a ventilation interface assembly according to an embodiment of the present invention when fastened to the face and head of a person, who also may be referred to as a patient or user, Pt. Ventilation interface assembly 50 includes a head strap assembly 80, a compound arcuately shaped manifold 60 beneath the nose N, a ventilation gas supply assembly 120 attached unilaterally to only one side of manifold 60 and positioned to only one side of the person's face, and two nasal cushions 70 extending superiorly from the manifold to impinge on, enter, and preferably seal with the person's nostrils. Nasal cushions may also be referred to as nasal sealing cushions. Gas supply assembly 120 is attached to a ventilator (not shown) for the supply of PAP to the person's nose and airway. Manifold 60 may preferably be designed with compound arcuate curves, which preferably function to optimally and comfortably match the person's anatomy, to increase comfort, and to help stabilize the manifold against the face when strapped in place by the head strap assembly. Nasal cushions 70 preferably may be designed with features that function to exert a light and forgiving, yet effective, sealing force around the rim of the nostrils. Nasal cushions 70 may be permanently or removably attached to the manifold. Manifold 60 and gas supply assembly 120 preferably may be designed such that air flow turbulence is minimized, thus creating a low pressure drop design, which preferably improves sealing efficacy and reduces noise and leaks. For example, because the airflow from the gas source preferably enters the manifold from only one side at any given time, the turbulent collision of air associated with gas flow into manifolds, tubes, or cannulae from both sides at once is preferably avoided. Head strap assembly 80 preferably may be designed with features, adjustments and materials that function to provide a secure, yet soft, comfortable and conformal fit to the user. Manifold 60 and head strap assembly 80 preferably possess shapes that enable any given user to place the structures in the most comfortable and unobtrusive parts of their face and head. The overall assembly preferably functions to permit the user to sleep in a variety of positions with comfort and without causing the mask to shift and malfunction. The user preferably can freely sleep on the side of their face opposite to the gas supply attachment. In addition, the user preferably can freely sleep on the side of their face to which the gas supply attachment is attached, for example by rotating the gas supply assembly inferiorly. In addition to preferably being comfortable in any sleep position and sealing effectively, preferable embodiments of the inventive ventilation interface assembly preferably present minimal obtrusiveness and annoyance to the user because the ventilation interface assembly preferably may be positioned such that the assembly is relatively away from the user's eyes, ears, and mouth, and the gas supply assembly 120 is completely away from one side of the face. Because preferable embodiments of this invention preferably allow the patient to sleep comfortably without obtrusiveness, the patient is preferably able to tolerate and comply with his or her prescribed PAP therapy, and therefore the medical treatment may be more efficacious when compared to using other PAP ventilation interface devices. With other PAP ventilation interface devices, it has been proven that the patient has poor tolerance for and compliance with the therapy because the interface device is too uncomfortable or too obtrusive, and the patient does not use it as prescribed and therefore essentially remains poorly treated or even untreated. It can be seen therefore that preferable embodiments of this invention preferably solve many of the previously unsolved problems of PAP interface devices, and especially those for treating OSA.

Numerous embodiments of manifolds are included in embodiments of the present invention. Preferably, more than one type and/or size is provided with a ventilation interface assembly according to an embodiment of the present invention, so that each user can select a manifold that fits comfortably. As another preferable alternative, a clinician may fit a user with an appropriate manifold or manifolds that fit(s) comfortably. As one non-limiting example, the user may find that a manifold having an axial arc length longer than the width of the base of their nose is most comfortable. Likewise, numerous embodiments of nasal cushions are included in embodiments of the present invention. Preferably, more than one type and/or size that are compatible with the manifold(s) provided is provided with a ventilation interface assembly according to an embodiment of the present invention, so that each user can select nasal cushions that fit comfortably. As another preferable alternative, a clinician may fit a user with appropriate nasal cushions that fit comfortably and that are appropriate with the manifold or manifolds that that are provided to the patient.

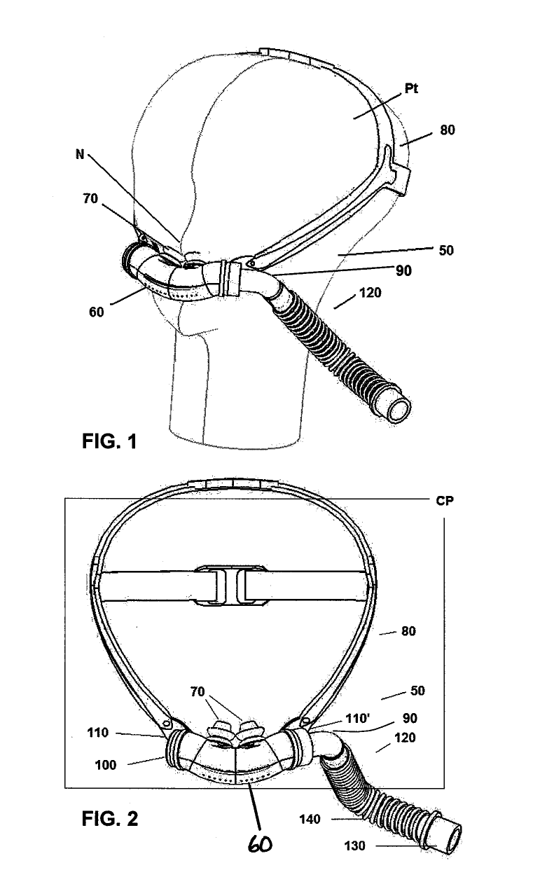

FIG. 2 illustrates a front or coronal plane CP view of an embodiment of a ventilation interface assembly 50 of an embodiment of the invention. Gas supply assembly 120 comprises a flex hose 140, a proximal end gas supply connector 130 to connect to a ventilator or ventilation gas delivery circuit, and a distal end swivel elbow connector assembly 90, which preferably connects gas supply assembly 120 to manifold 60. The ventilator or ventilation gas delivery circuit is not shown. With this and other embodiments of the invention, any suitable ventilator or gas delivery circuit may be used. Throughout the present disclosure, the terms distal and proximal are relative positional terms, where proximal refers to a position relatively closer to the ventilator or ventilation gas circuit, and distal refers to a position relatively farther from the ventilator or ventilation gas circuit. “A” is used, particularly in the figures, to refer to the front/anterior side of a manifold. “P” is used, particularly in the figures, to refer to the back/posterior side of a manifold. “S” is used, particularly in the figures, to refer to the top/superior side of a manifold. “I” is used, particularly in the figures, to refer to the bottom/inferior side of a manifold. Swivel elbow connector assembly 90 preferably may be used to rotate or swivel gas supply assembly 120 to the most desirable position by the user. The side of manifold 60 opposite to swivel elbow connector assembly 90 is preferably occluded with an end cap 100. Preferably, manifold 60, swivel elbow connector assembly 90, and end cap 100 are configured such that the clinician or patient can switch the side of manifold 60 that is connected to each of swivel elbow connector assembly 90 and end cap 100. As another preferably alternative, the clinician or patient may be provided with a kit that contains at least two manifolds: one with swivel elbow connector assembly 90 on the left and end cap 100 on the right, and another with swivel elbow connector assembly 90 on the right and end cap 100 on the left. Manifold 60 preferably is held in place by head strap assembly 80; head strap assembly 80 and manifold 60 are preferably connected together with left and right interconnecting assemblies 110′, 110, which preferably are constructed and/or assembled to allow for a moveable or adjustable attachment between head strap assembly 80 and manifold 60, so that the ventilation interface assembly preferably can be optimally adjusted to fit the individual user. Of course, manifold 60, end cap 100, and/or gas supply assembly 120, as well as other embodiments of manifolds and connector assemblies, may be used with other devices for securing them to the patient's head, face, and or nose.

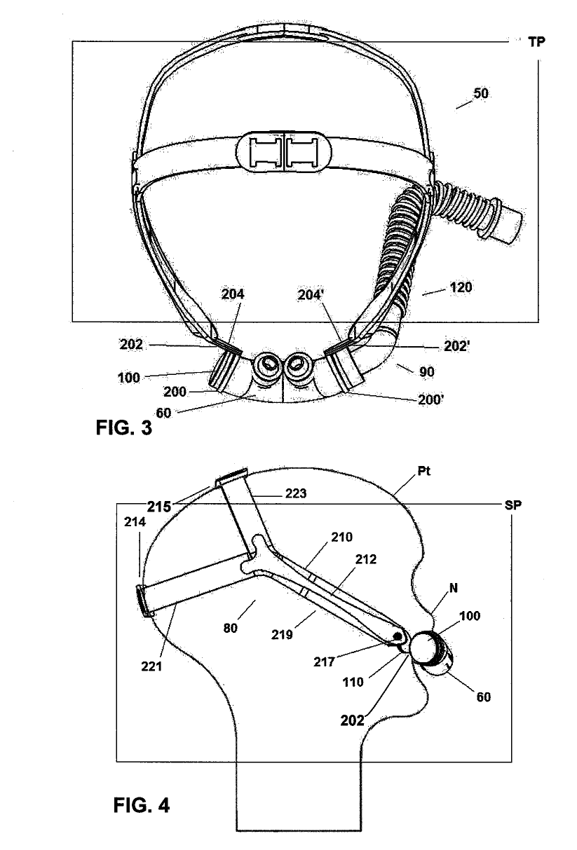

FIG. 3 illustrates an embodiment of ventilation interface assembly 50 of an embodiment of the invention in the top view or in the transverse plane TP. Left and right interconnecting assemblies 110′, 110, preferably each comprise a left and right manifold connecting ring 200′, 200, a left and right head strap attachment plate 202′, 202, and an attachment plate skin pad 204′, 204. In the embodiment of manifold shown, the posterior sweeping curve of the manifold's compound arcuate curves, which preferably helps position manifold base 60 comfortably and effectively against the face, is shown.

FIG. 4 illustrates a right side or sagittal plane SP view of an embodiment of a patient ventilation interface assembly 50 on a person Pt, showing in more detail the components of an embodiment of head strap assembly 80. Head strap assembly 80 includes left and right forward head straps 219′, 219 (left forward head strap 219′ not shown), and at least one rear strap 221 with a rear strap buckle 214, and optionally a second, top head strap 223 with a top strap buckle 215, and left and right head strap-manifold joints 217′, 217 (left head strap-manifold joint 217′ not shown). Forward head straps 219′, 219 preferably each include a soft fabric or elastomer strap material 210′, 210 and a stiffening member 212′, 212 (left strap material 210′ and stiffening member 212′ not shown). In a preferable embodiment, joint 217′, 217 between head strap assembly 80 and manifold 60 (left joint 217′ not shown) is adjustable. Forward strap stiffening member 212′, 212 and head strap attachment plate 202′, 202 may be interconnected with a hole and protuberance connection on the mating components. For example, a hole can be provided near the anterior end of stiffening member 212′, 212, and a barbed protuberance can be provided near the posterior end of attachment plate 202′, 202, in which case stiffening member 212′, 212 would be snapped over attachment plate 202′, 202. As another example, a barbed protuberance can be provided on stiffening member 212′, 212 and a mating hole on attachment plate 202′, 202, in which case attachment plate 202′, 202 would be snapped in place over stiffening member 212′, 212. The hole and protuberance connection preferably provides a swiveling or pivoting motion to allow the joint to pivot, preferably allowing the overall ventilation interface assembly 50 to be configured and adjusted mate well to the individual's anatomy. Alternatively, the hole and protuberance connection can include multiple discrete settings, to that the user or care provider can adjust the angle of the joint as desired. In this case, a series of channels, ribs, or key ways may be provided in attachment plate 202′, 202 and stiffening member 212′, 212 to create different angle settings. Stiffening member 212′, 212 and attachment plate 202′, 202 preferably may be comprised of a semi-rigid thermoplastic material, such as urethane, PVC, polyethylene, polypropylene or polysolfone, in order to provide the preferred mechanical strength for the joint. Preferably, the joint strength may be 0.1-0.5 pounds for connecting force tension and up to 10 pounds for withstand pressure exerted on the joint by the patient's weight. More details concerning these components, and other types of joints that are included in certain embodiments of the invention, are described herein.

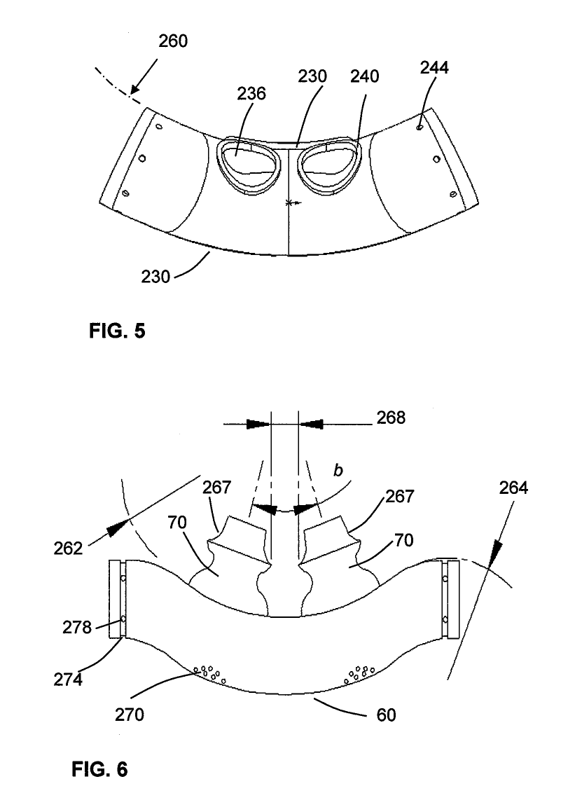

FIGS. 5-15 illustrate embodiments of manifolds according to some embodiments of the invention in more detail.

FIG. 5 illustrates an isometric top view in the transverse plane of manifold 60. In this embodiment, the manifold shape curves laterally from the midline with a lateral-posterior sweeping curve 260 to preferably match the shape of the face under the nose, and is preferably also curved upward or superiorly, as is depicted in other views. The manifold preferably includes attachment rims 240 for attaching nasal cushions, which preferably may be removably attachable, and gas flow openings 236 that allow passage of ventilation gas through the nasal cushions. Nasal cushions may also be permanently attached to manifold 60, either to attachment rims 240 or by other means. FIG. 5 also illustrates manifold connecting ring attachment holes 244, for mating and attachment of manifold-head strap attachment connecting rings 200′, 200 (not shown) and for connecting an end cap or gas supply assembly. Optionally, manifold 60 can include a skin pad 230 on the posterior skin side, which can preferably help tilt the angle of the manifold into the correct angle in the sagittal plane so that the nasal cushions, when attached, are aligned with the angle of the nostril foramen. If provided, pad 230 can also absorb strapping forces.

The manifold lateral posterior curve 260 is preferably a 1.0-5.0″ radius, more preferably: 2.0-4.0″ for adult sizes, 1.0-2.0″ for pediatric sizes, and 0.5-1.0″ for neonatal sizes. The manifold 60 may be provided in several cross sectional outer dimension sizes such as, for non-limiting example: 12-16 mm for a small size, 14-18 mm for a medium size, 16-20 mm for a large size, 10-14 mm for an extra small size, and 18-22 mm for an extra large size. The manifold 60 may be provided in at least three left to right length sizes such as, for non-limiting example: 3-2.5 inches for a large size, 2.5-2 inches for a medium size, and 2-1 inches for a small size. Manifold 60 may preferably be comprised of an elastomer, such as silicone, urethane, Santropene, or elastomer blends, such as a urethane-PVC blend, or C-Flex. Manifold 60 can also be made of a thermoplastic elastomer, or a plasticized thermoplastic. The material hardness of manifold 60 is preferably 40-60 Shore A, and it's wall thickness is preferably 0.040-0.120″. Manifold 60 can also be constructed of a material that can be re-formed or reshaped by the caregiver or end user in order to better mate with the end user's anatomy. Reshaping of manifold 60 can also be made possible by fabricating it with a thermoset material that can be heated and reformed, such as PVA, or by including malleable members within the construction of the manifold to enable the patient or clinician to bend and flex it into the shape desired as will be described subsequently.

FIG. 6 illustrates a front view of manifold 60 of FIG. 5, with nasal cushions 70 attached to manifold 60. Manifold 60 includes a lateral-superior sweeping curve 262, which levels off to a lateral sweeping curve 264 near the lateral ends. The lateral superior curve is preferably a 1.0-3.0″, radius and more preferably a 1.5-2.5″ radius, for adult sizes. Curves 262 and 264 are preferably compounded with a posterior sweeping curve 260 (shown in other views, for example FIG. 5). Such compound arcuate curves preferably allow the manifold to better match the anatomy of the face, such as avoiding obstructing of the mouth, and avoiding being placed at uncomfortable structures of the face, such as the cheek bones and jaw bones. In addition, the curves can serve to angulate the distal ends of the nasal cushions inward in the coronal plane. The angle b between nasal cushions 70 is preferably 20-60 degrees, more preferably 30-50 degrees, and even more preferably 36-48 degrees; and the spacing 268 between sealing surfaces 267 of the nasal cushions is preferably 0-7 mm, and more preferably 2-5 mm. However, other options for the attachment, spacing, and angulation of nasal cushions are possible and included in some embodiments of the present invention. For example, the nasal cushions also may include flex features to allow them to flex and compress, as is described in more detail herein. FIG. 6 also illustrates a manifold attachment ring groove 274 and manifold connecting ring attachment holes 278 for mating and attachment of the manifold-head strap attachment connecting ring 200′, 200 (not shown) and for connecting an end cap or gas supply assembly. The groove preferably allows the attachment connecting ring 200′, 200 to be attached to the manifold with a flush surface. The groove is preferably 0.030-0.090″ deep and 0.080-0.250″ wide.

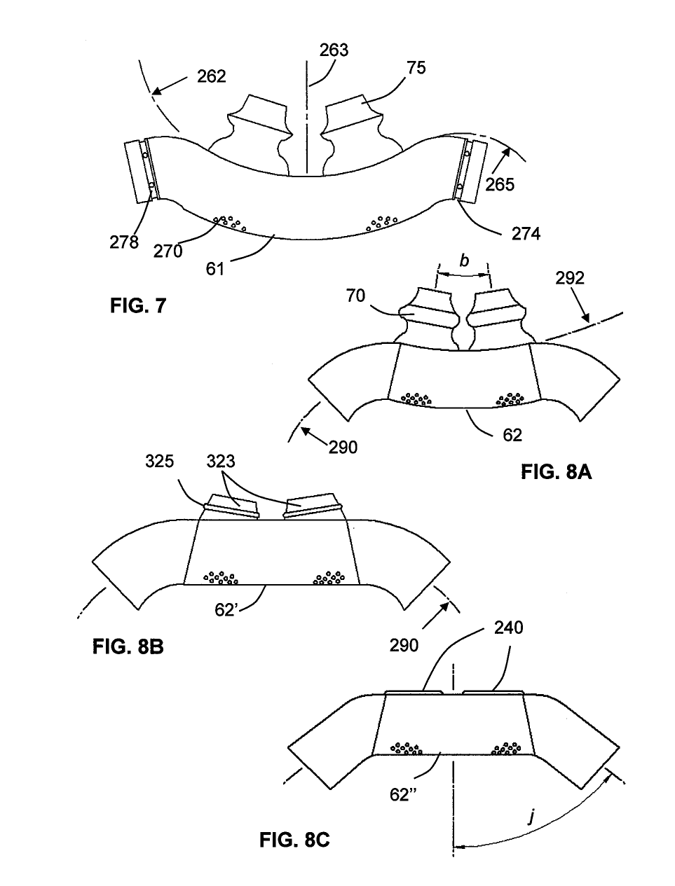

FIG. 7 illustrates a front view of an alternative embodiment of a manifold according to an embodiment of the present invention. In FIG. 7, manifold 61 comprises compound arcuate curves on the left and right side of the manifold, wherein the compound arcuate curves comprise a lateral-superior first curve 262 extending bilaterally from the manifold midline 263, and transitioning to a lateral-posterior inferior second curve 265 extending to the lateral ends of the manifold. Also illustrated are some additional features which may optionally be used with manifolds in accordance with an embodiment of the invention.

Optional exhalation exhaust ports 270 are illustrated in FIG. 7. In the embodiment of FIG. 7, exhalation exhaust ports 270 are two groupings of multiple holes through the inferior wall of the manifold positioned such that each grouping is approximately opposite to a nasal cushion 75, so that exhalation exhaust ports 270 are aligned with the direction of exhaled flow through the cushions, and misaligned with the direction of inspired flow flowing into the manifold from the left or the right and upward into the nasal cushions. Therefore, exhalation through exhalation exhaust ports 270 preferably may be relatively easy and leakage through exhalation exhaust ports 270 during inspiration preferably may be relatively minor. The diameter of each of exhalation exhaust ports 270 is preferably 0.5-3.0 mm, and the total combined cross sectional area of exhalation exhaust ports 270 is preferably 50-225 mm². The combined resistance of exhalation exhaust ports 270 is 2-20 cmH₂O/L/sec. The entrances of exhalation exhaust ports 270 on the inside of the manifold preferably may be rounded to reduce resistance to gas flow exiting through the ports from the inside of the manifold. The pattern of exhalation exhaust ports 270 may optionally simulate the shape of the nares so that the vented gas approximates the flow pattern created from exhaling through the nose. Exhalation exhaust ports 270 may optionally be oriented on a diagonal through the manifold wall, oriented in a tighter pattern on the inside of the manifold, and fanning out to a larger pattern on the outside of the manifold so that the vented gas fans out similar to exhaling from the nose. Optionally, exhalation exhaust ports 270 may have a one-way valve feature to allow flow in the exhaled direction, but restrict flow in the inspired direction. Exhalation exhaust ports may optionally be placed in other locations and patterns, such as, for non-limiting examples, across the entire inferior surface of the manifold, in the middle of the inferior surface of the manifold, or on the inferior surface of the manifold from a location below one nasal cushions to below the other nasal cushion, or combinations of locations. Exhalation exhaust ports may have circular cross-sectional shapes or other cross sectional shapes, such as oval, triangular, or rectangular.

FIG. 7 also illustrates a manifold attachment ring groove 274 and connecting ring attachment holes 278 for mating and attachment of manifold-head strap connecting rings 200′, 200. The groove preferably allows connecting rings 200′, 200 to be attached to the manifold with a flush surface. The groove is preferably 0.030-0.090″ deep and 0.080-0.250″ wide. Optionally, the connecting rings can be permanently affixed to the manifold, rather than removably attachable. In this later case the ring can be bonded to the manifold, or the manifold can be molded around the ring. FIG. 7 also depicts an optional embodiment in which the nasal cushions 75 can be pre-attached or permanently fixed to the manifold, by being bonded to the manifold, or molded with the manifold.

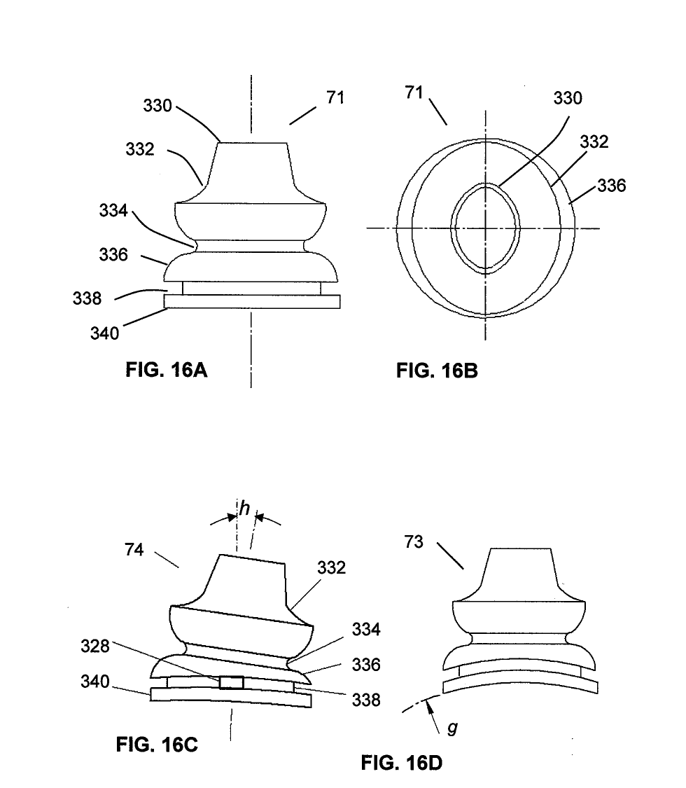

FIGS. 8A-8C illustrate alternate embodiments of the manifold's compound arcuate curves in which the manifold curves laterally, posteriorly, and inferiorly. FIG. 8A illustrates a front view of manifold 62 in which the manifold comprises compound arcuate curves with a left and right lateral-posterior-superior curve 292 and a lateral-posterior-inferior curve 290 sweeping to the left and right lateral ends of the manifold. Curve 292 is preferably a 3.0″-8.0″ radius, more preferably a 4.0″-6.0″ radius. This shape preferably positions the manifold against the soft structures of the face, lateral to the nose, most preferably between the cheek bone and jaw bone and preferably helps angulate nasal cushions 70 inward toward the midline to match the anatomy of the nostrils. Optionally, the center of the manifold near the midline 263 can include an inferior curve. The superior part of the lateral-posterior-superior curve 292 can be a substantial curve, such as 1.0-3.0″ radius, or can be a gradual curve, such as a 3.0-5.0″ radius. Alternatively, this section can be straight or have a superior curve, can be angled, or combinations thereof FIG. 8B illustrates an alternative manifold geometry similar to the manifold in FIG. 8A; manifold 62′ comprises a straight surface in the coronal plane near the midline. FIG. 8B also illustrates an alternative embodiment in which bosses 323 are attached to the superior surface of the manifold 62′; nasal cushions may be attached to the bosses. The bosses can include a connecting ring or ridge 325, which facilitates secure attachment of nasal cushions. The bosses and/or cushions preferably may be angulated with respect to the manifold superior surface, to facilitate proper angulation of the nasal cushions in the coronal plane to match the angle of the nostrils. The bosses and/or cushions optionally may also be angulated in the sagittal plane, to facilitate proper angulation of the nasal cushions in the saggital plane to match the angle of the nostrils. Additional optional details regarding the nasal cushion bosses are illustrated in FIG. 15A. FIG. 8C illustrates an alternative embodiment to the manifold shape described in FIG. 8B, in which the manifold 62″ comprises a lateral-posterior curve, not seen in the view shown, and lateral-inferior angles instead of a curve. The lateral-inferior angle j may preferably be 25-75 degrees, more preferably 40-60 degrees. In the embodiment illustrated in FIG. 8C, the middle section superior surface of the manifold is shown as straight surface with attachment rims 240 thereon. In such embodiments, nasal cushions may preferably be adapted to attach to attachment rims 240, and attachment rims 240 optionally may be shaped to angulate the distal tips of the nasal cushions inward toward the midline, such as illustrated in FIG. 16C.

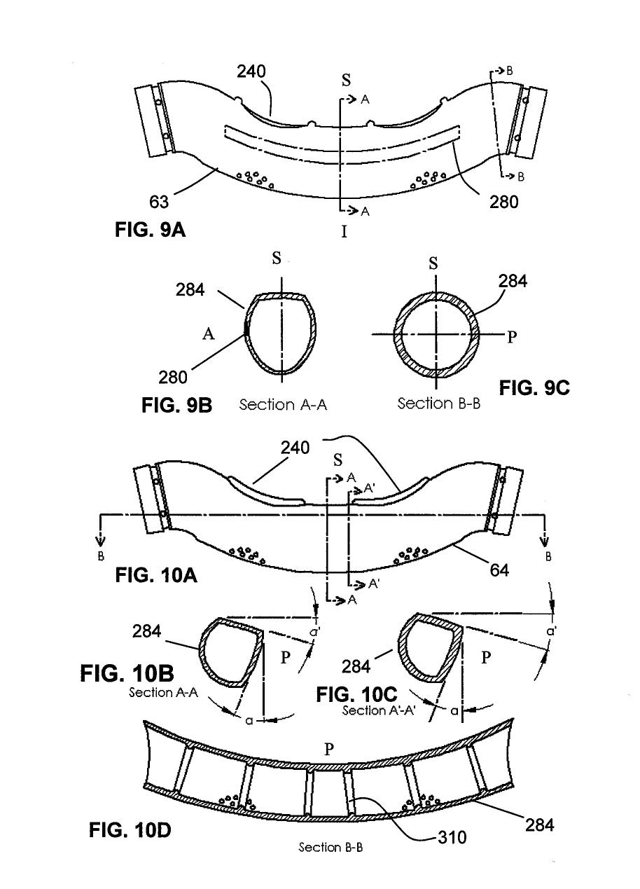

FIGS. 9A-9C illustrate a front view and cross sections of an alternative embodiment of a manifold 63 that includes a varying cross section and a stiffening member. FIG. 9B illustrates a cross section through the middle section of the manifold at line A-A of FIG. 9A. The cross sectional dimension in the anterior-posterior axis may be shorter than in the inferior-superior dimension, which may reduce the profile of the manifold protruding from the users face. The curvature of the top superior surface S of the manifold may be flattened, which may provide a flatter connecting surface for attachment of the nasal sealing cushions. FIG. 9C illustrates the cross sectional profile of manifold 63 at line B-B near the lateral ends of the manifold, which may be substantially circular, which may provide a suitable attachment for round connectors or caps to the ends of the manifold. As another non-limiting alternative, the non-round cross-sectional geometry may continue through the manifold length to the manifold ends, and when connecting rigid round connectors to the manifold, the manifold material may conform to the rigid materials due to the silastic properties of the preferred manifold materials. The manifold may also include other features described herein, such as attachment rims 240 to which nasal cushions may be attached, and gas flow openings 236 for passage of gas to the nasal cushions (not shown), and attachment ring connecting features, among other features.

FIGS. 9A and 9B also illustrate an optional stiffening member 280, which may be attached to the anterior (A) wall of the manifold 63. The stiffening member 280 preferably may be placed inside the anterior wall, for example during molding of the manifold; however it may alternatively be placed on the outside wall, on the inside wall, or in or on other walls, such as the inferior, superior, or posterior wall. Manifold 63 may optionally comprise multiple stiffening members. A stiffening member 280 may be, as a non-limiting example, a strip of material, preferably with a thickness of 0.005″-0.030″, more preferably 0.010″-0.020″, and preferably having a width of 0.040″-0.200″, more preferably 0.060″-0.130″. A stiffening member 280 may possess a rectangular, oval, round, straight, or curved cross section. The stiffening member 280 preferably may be comprised of malleable material, such as a brass, copper or nickel alloy, which preferably can be shaped by the user or clinician to adjust the compound curvature of the manifold so that the manifold can be further customized to mate with individual anatomies. As other non-limiting alternatives, the stiffening member may be comprised of a shape memory material, such as a aluminum alloy, a thermoplastic resin such as ULTEM, or a spring steel, or a shape memory metal such as Nitinol. As another alternative, the manifold may be stiffened by a framework or superstructure of thermoplastic struts, in which case the manifold may be manufactured, for example, by a two step molding process: a first step of molding the thermoplastic framework, and a second step of molding a silastic material around the framework. The framework may be comprised of, for example, a thermoplastic, for example polypropylene, polysolfone, nylon or Ultem; and the surrounding material may be comprised of an elastomer, for example silicone, urethane or Santropene. The framework preferably comprises 10-25% of the overall material volume of the manifold, and the elastomer surrounding preferably comprises the balance of the material volume.

FIGS. 10A-10D illustrate an alternative embodiment of a manifold according to an embodiment of the present invention, with manifold 64 comprising a non-circular central cross section and stiffening ribs within the cross sectional profile. FIG. 10A illustrates a front view of the manifold 64 with compound arcuate curves, including a lateral-superior curve, and a lateral inferior curve. However, these curves are exemplary, and other compound arcuate or angled curves, such as those described elsewhere in this specification, also are included in this embodiment. FIG. 10B illustrates a cross section through the anterior-posterior plane at line A-A at or near the midline 263 of the manifold shown in FIG. 10A, and indicates the manifold cross sectional profile and geometry of the manifold walls 284. The cross section shows that the posterior (P) wall of the manifold may be angled with an angle a, and the superior (S) wall of the manifold may be angled with an angle a′. The angle a preferably matches the anatomical angle of the user's face between the nose and mouth, and the angle a′ provides a tilted superior surface, which preferably facilitates preferable positioning of nasal cushions, preferably such that the nasal cushions extending from the superior surface of the manifold are angled in the sagittal plane so that they are aligned with the angle of the nostril foramen. The cross section at or near the lateral ends of the manifold is preferably substantially round (not shown) to facilitate convenient attachment to a round plug or gas supply hose connector, as described herein. Therefore, the manifold cross section preferably may transition from a non-round cross sectional profile in the middle section of the manifold, to a substantially round cross section at the ends.

Optionally, as illustrated in FIG. 11, a skin pad 230 can be attached to the posterior side of the manifold. FIG. 11 illustrates an alternate cross section of the medial section of the manifold in FIG. 10A, illustrating an optional skin pad attached to the posterior side of the manifold. Skin pad 230 preferably may aid in the control of the angle a, and in absorbing the strapping forces exerted by the head strap assembly preferably used to secure the manifold to the face. Angle control may be performed by the shape of the skin pad or the compressibility of its material. The skin pad 230 can be removable or fixed, and may preferably be comprised of a compressible shape memory material, such as a viscoelastic material, for example shape memory foam, or a silicone gel. The skin pad can be removably attached to the manifold, or can be an extension of the manifold material. In some embodiments, such as wherein the skin pad is removable, multiple skin pads may be provided to the clinician or user. The multiple skin pads may be of different shapes, sizes, and compressibilities, which preferably may allow the user to choose the most comfortable skin pad. The multiple skin pads may be of the shape, size, and compressibility, which preferably may allow the user to change the pad, should it become soiled or worn.

FIG. 10C illustrates a cross section at line A′-A′ near the midline of the manifold shown in FIG. 10A, at a distance toward a lateral end from the cross section A-A, wherein the manifold wall 284 may be thickened due to the presence of a radial stiffening rib 310 on the inner wall of the manifold in that location. FIG. 10D illustrates a left-to-right cross section of the manifold shown in FIG. 10A at line B-B which illustrates multiple radial ribs 310. Such radial stiffening ribs can be spaced evenly or in a pattern throughout all or part(s) of the manifold length. Ribs 310 preferably stiffen the manifold, which is preferably constructed of a soft material, preferably an elastomeric material. The ribs 310 thus preferably resist collapse or compression of the manifold's structure, while still maintaining the softness and comfort of the manifold. The ribs 310 preferably protrude inward from the inside surface of the manifold, preferably have a height of 0.040-0.100″, and preferably are 0.030-0.125 in width. The spacing between the ribs preferably is 0.25-0.75″.

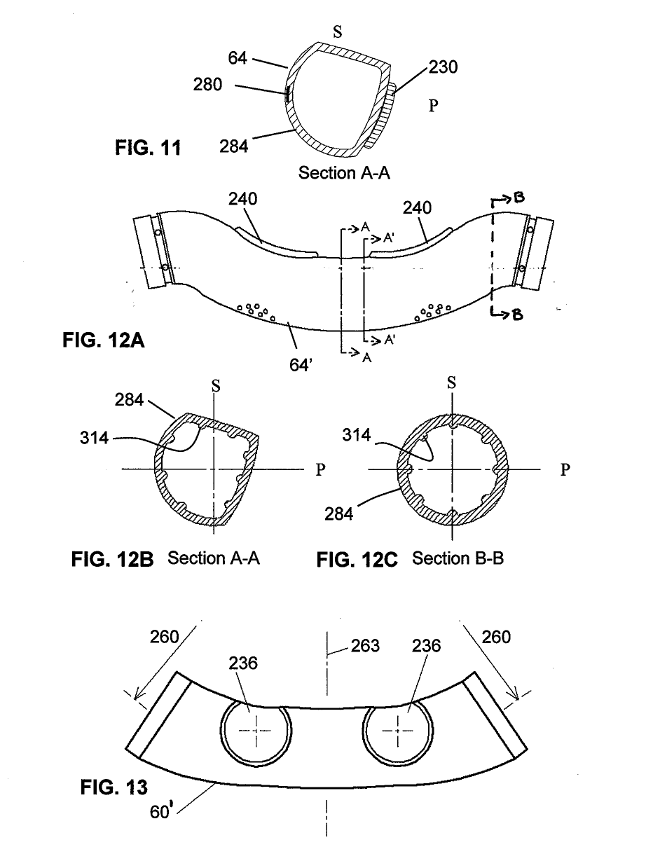

FIGS. 12A-12C illustrate an alternative embodiment of a manifold 64′ in accordance with an embodiment of the present invention, in which axial, or longitudinal, stiffening ribs 314 are provided in the construction of the wall of the manifold. Such axial ribs 314 can be spaced evenly or in a pattern throughout all or part(s) of the manifold wall. Axial ribs 314 may optionally be used in combination with radial ribs 310. FIG. 12B illustrates a cross section near the midline of the manifold at line A-A shown in FIG. 12A. The cross section shows a pattern of axial ribs 314 constructed into the wall 284 of the manifold 64′. FIG. 12B also illustrates a non-round cross section of the manifold, which preferably facilitates mating with the angle of the patient's face and alignment of the nasal cushions to the nostril foramen angle. FIG. 12C illustrates a cross section near the lateral end the manifold in FIG. 12A at line B-B, with ribs 314. The non-round cross section illustrated in FIG. 12B preferably transitions to a substantially round cross section, as shown in FIG. 12C. The substantially round cross section preferably facilitates connection to a manifold end cap or gas supply connector. Optionally, the ribs can be discontinued near the lateral ends of the manifold, to provide a smooth round surface for mating of the manifold end cap and gas supply connector; as another non-limiting alternative, the ribs can continue to the manifold lateral ends, and the end cap and gas supply connector can have mating geometry with grooves to align with and slide into the ribbed manifold geometry. A rib or ribs may also be intermittent along the length of the manifold, with sections of rib alternating with smooth sections. The axial ribs indicated in FIGS. 12A-12C may be preferred over the radial ribs indicated in FIGS. 10A-10D since the axial ribs may cause fewer disturbances to the laminar gas flow profile within the manifold. The axial ribs are preferably 0.040-0.100″ in height, are preferably 0.030-0.125″ wide, and are preferably spaced 20-45 degrees apart.

FIG. 13 illustrates a top view of an embodiment of a manifold in accordance with an embodiment of the invention, in which the manifold 60′ is similar to the manifold of FIG. 5, including left and right lateral-posterior curves 260 sweeping from the midline 263 to the left and right lateral ends. The center section near the midline 263 between the gas flow openings 236 can include a straight section as shown, or alternatively the left and right lateral-posterior curves 260 can start at and include the midline. In the manifold 60′ of FIG. 13, the center section of the manifold near the midline is less wide in the anterior-posterior dimension compared to the width of the manifold width at the distal ends; such a shape preferably reduces the profile of the manifold and obtrusiveness under the nose. Alternatively, the manifold can be wider in the anterior-posterior dimension in the center section to facilitate accommodation of the nasal cushion attachment, and less wide lateral to the nose; this shape preferably reduces profile and obtrusiveness lateral to the nose.

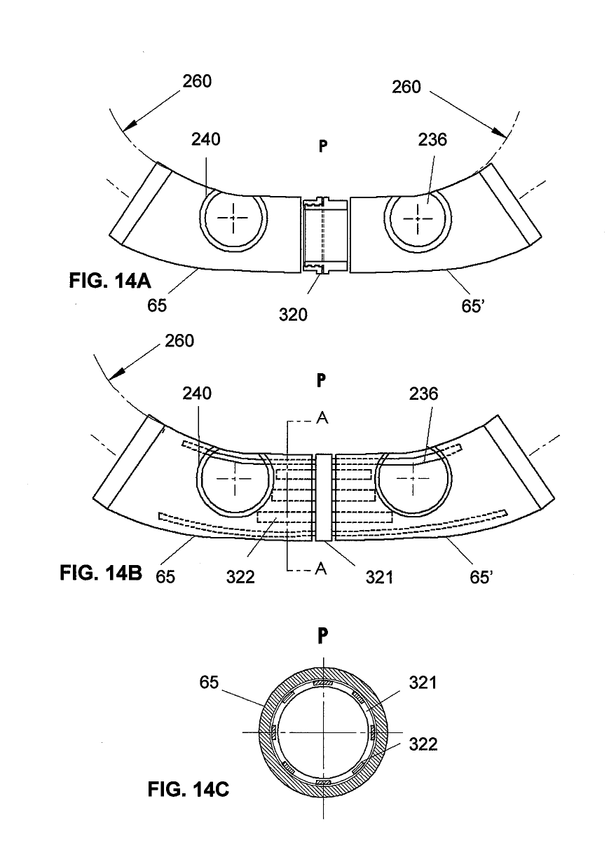

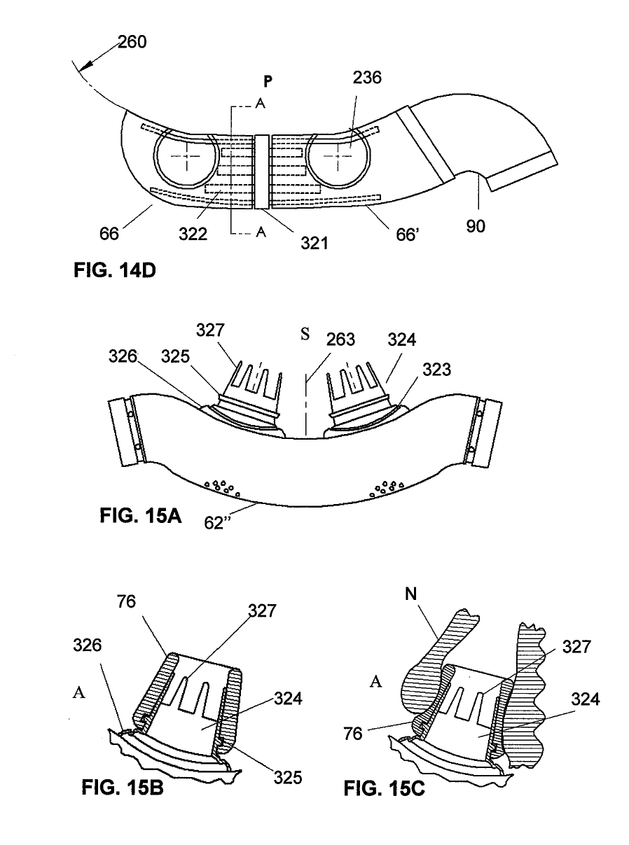

FIG. 14A illustrates a top view of an optional embodiment in which the manifold is a two piece manifold, with a left piece 65′ and a right piece 65, and a manifold central connecting ring 320 interconnecting the manifold pieces 65′ and 65. The manifold central connecting ring 320 preferably allows the user or clinician to adjust the overall length of the assembled manifold, so that the spacing of the nasal cushions, when attached, more closely matches the spacing of the user's nostrils. The manifold central connecting ring 320 may be comprised, for example, of a left and a right mating piece that are fitted together with a light interference fit, in which the two pieces can slide in and out of each other to adjust the length of the two piece ring. The two pieces may include mating detent features to set multiple positions and resist inadvertent adjustment. Other length-adjustable manifold central connecting rings are contemplated and can be used with the embodiments of the present invention. Non-limiting examples include manifold central connecting rings comprised of a left and a right mating pieces that twist together to varying lengths and a single piece connector, wherein the manifold left piece 65′ and manifold right piece 65 can be slidably moved over the connector. The left and right manifold pieces 65′, 65 can be un-removably attached to the left and right ends of the manifold central connecting ring during manufacturing, or during dispensing to the end user, so there is less assembly required by the end user. In such embodiments, the overall length of the manifold assembly may be adjusted by adjusting the length of the manifold central connecting ring. Alternatively, the manifold central connecting ring may be adjustably attached to one or each of the left and right manifold pieces 65′, 65, and the overall length of the manifold assembly may be adjusted by adjusting the engagement length of the connections between the left and/or right manifold pieces 65′, 65 and the manifold central connecting ring. In such embodiments, the length of the manifold central connecting ring can itself be non-adjustable or adjustable. Alternatively, multiple sizes and lengths of the manifold central connecting ring can be made available so that the user can select a preferable size and length. If multiple sizes of manifold central connecting rings are made available, they may themselves have adjustable or non-adjustable lengths, and may be adjustably or non-adjustably connected to the manifold pieces 65′, 65. The connecting ring is preferably comprised of a thermoplastic, such as polypropylene, polyethylene, polysolfone, nylon or PVC. Optionally, the outer diameter of the connecting ring can include an elastomer covering to provide a soft contact to the user.

FIG. 14B illustrates a top view of an optional two piece manifold embodiment. A left and right manifold piece 65′, 65 are connected by a manifold central connecting member 321, which preferably performs one or both of the following two functions. First, the connecting member 321 is preferably usable to adjust the distance between the nasal sealing cushions, so that the distance more closely matches the nares of the individual user. Second, the connecting member preferably includes structural connecting member struts 322, which preferably extend into the inside of the manifold pieces 65′, 65, thereby preferably providing structural rigidity to the manifold, which is preferably made of a pliable material. FIG. 14C illustrates a cross section through the manifold assembly of FIG. 14B, indicating the connecting member 321, the connecting member framework struts 322, and the manifold 65. The connecting member 321 preferably extends into the manifold far enough to effect a seal, for example 0.100-0.200″ into the manifold. Connecting member 321 is preferably comprised of a thermoplastic, such as polypropylene, polyethylene, polysolfone, nylon or PVC. The ring portion 321 is preferably 0.020-0.080″ thick; and the struts 322 are preferably 0.015-0.050″ thick, 0.040-0.120″ wide, 0.5″-1.5″ long, and spaced at 20-45 degree increments. A single device may comprise struts 322 of varying lengths.

FIG. 14D illustrates an alternate embodiment of a manifold in accordance with an embodiment of the present invention. In the manifold of FIG. 14B, the left and right manifold pieces 66′, 66, are not symmetric with each other. One side is configured to be tapered closed, and the other side is configured for attachment of swivel-elbow connector assembly 90. As illustrated, a central connecting member 321 may be used to removably attach the left and right manifold pieces. Through the use of connecting member 321, the side of attachment of the gas supply attachment can be changed. Alternatively, a central connecting ring 320 may be used, or the manifold may be in a single piece. A kit can be provided to the clinician or user, which includes two left side pieces, one closed and one opened, and two right side pieces, one closed and one opened. An alternative kit (or the same kit) could include a single-piece manifold with closed right end and a single piece manifold with a closed left end. Also, multiple sizes of diameters and lengths of manifold pieces 66′, 66 can be provided to give the clinician or end user additional fitting range options.

FIG. 15A illustrates an alternative embodiment of a manifold in accordance with an embodiment of the present invention. In FIG. 15A, a manifold 62″ includes raised attachment bosses 324 for attachment of nasal cushions. Nasal cushions, non-limiting examples of which are shown in FIGS. 15B and 15C, preferably may be removably attached to the attachment bosses. The bosses may extend, for example, for 50% of the length of the nasal cushion; and/or, as shown, may include extensions 327 extending close to the full length of the nasal cushion. Preferably, the boss or boss extensions are more rigid than the nasal cushion, so that the boss or boss extensions provides a structural frame or support for the cushion. Nasal cushions preferably may be extremely soft, and conformable for contacting, conforming to and sealing against the nostril surfaces without exerting uncomfortable forces. For example, the attachment boss or extension may preferably posses a compression strength of 50% radial compression at 0.05-1.0 pounds radial force, or more preferably 0.1-0.5 pounds radial force. In some embodiments in which bosses 324 are used, the nasal cushions can preferably be comprised of even softer material than if the bosses were not used. For example, nasal cushions, particularly if used with bosses, may be of a durometer of 10-50 Shore OO. Such very soft nasal cushions may also be referred to as “nasal cushion sleeves” and are preferably used with bosses; likewise, bosses are preferably used with nasal cushion sleeves. For example, nasal cushions, particularly if used with bosses, may also be very compressible; for example nasal cushions may preferably radially compress 50% of their wall thickness with 0.05-0.25 lbs of radial force. The nasal cushions may preferably be comprised of a viscoelastic material with fast dimensional recovery properties, with a non-limiting example being a shape memory foam with a greater than 80% recovery from 25% compression in less than 3 seconds. FIG. 15B illustrates a cross section through an exemplary nasal cushion 76 preferably having a width dimension 10-50% larger than the sealing dimension, more preferably 20-30% larger, for example 6 mm-30 mm wide. FIG. 15C illustrates the nasal cushion of FIG. 15B when in use, indicating the compression of the sealing area to the sealing dimension to conform to the anatomy of the nostril. The nasal cushion and nasal cushion boss may include a mating feature, which may prevent, or lessen the likelihood of, the cushion inadvertently separating from the boss. A non-limiting example of such a mating feature is a retaining ring 325 on the boss, such as is illustrated in FIGS. 15A-15C. Another non-limiting example of such a mating feature is a groove on the boss and a protrusion or tensioning ring on the nasal cushion, which engages with the groove on the boss. Also shown in FIG. 15A is an optional flex feature at the base of the attachment boss 324. The flex feature preferably includes a pillow 326 and a groove 323, which preferably allow the nasal cushion to flex laterally, to flex posteriorly-anteriorly, and/or to compress superiorly-inferiorly. This ability to flex preferably allows the nasal cushion to flex and move to (a) allow minor alignment adjustments so that the cushions fit more correctly and comfortably with the nostrils, and (b) to facilitate maintenance of contact with the nostrils when the ventilation interface assembly experiences slight shifts in position when the user moves their head during use.

FIGS. 16A-18B illustrate some embodiments of nasal cushions in accordance with some embodiments of the invention in more detail.

FIGS. 16A and 16B illustrate an embodiment of a nasal cushion in accordance with an embodiment of the invention. The nasal cushion 71 of FIGS. 16A and 16B comprises an oval distal end and a round proximal base. FIG. 16A illustrates a front or end view, and FIG. 16B illustrates a top view. The oval distal end section includes a distal tip 330, and a sealing surface 332 that preferably seals on the nostril rim. The sealing surface may have a curved profile as shown in FIG. 16A. As another alternative, the sealing surface may have a stepped geometry which transitions from a first diameter to a second diameter in a step profile (not shown). As other alternatives, the sealing surface may be angled (not shown) or have another suitable geometry that allows for sealing with the nostril. The midsection of the nasal cushion can include an inward curvature or waist 334, which preferably facilitates flexing of the cushion to facilitate alignment with the nose and adjustment in response to movement of the ventilation interface assembly. Further, the cushion can include a flexible base pillow 336, which preferably can be compressed to help absorb forces of the manifold and cushion assembly pressing superiorly against the nose, so that the actual pressure against the nose is limited, and excess pressure is preferably absorbed by the base pillow 336. The cushion round proximal base may include a groove 338 to facilitate mating with an attachment rim, or other mating feature, and gas flow opening on the superior surface of a manifold. Groove 338 may preferably be round in order to facilitate rotation of the cushion relative to the mating feature of the manifold, so the clinician or user preferably can rotationally align the cushion to better match the user's individual anatomy. For example, one individual's anatomy may prefer that the long dimension of the oval tip be aligned in an anterior-posterior alignment, while another individual's anatomy may prefer that the long dimension of the oval tip be aligned side to side. The groove 338 and mating attachment ring or other mating feature on the manifold may optionally have positioning features so that the cushion can be set at discrete positions, and can better resist inadvertent rotational movement or incorrect rotational setting. The proximal base also may include a, preferably round, retaining flange 340 that can be inserted through the gas flow opening in the manifold to the inside of the manifold, preferably in order to better secure the nasal cushion to the manifold. The sealing surface 332 may have an oval cross section. The oval shape of the distal tip 330 may gradually transition to a round shape between the sealing surface 332 and the round attachment groove 338. Alternatively, the transition may be abrupt or step-wise. The oval distal end of the nasal cushions is one non-limiting example of nasal cushions according to some embodiments of the invention, and other shapes are also included in some embodiments of the invention, such as nasal cushions that are substantially round throughout their length, or nasal cushions that are substantially oval throughout their length.

Nasal cushions may preferably comprise an effective diameter dimension at the sealing surface, in free state, preferably of 1-3 mm greater than the effective diameter of the nostril opening of the user. Nasal cushions may be provided in various sizes. A given user may be provided with more than one size, or may be fitted with a size by the clinician. Exemplary preferred effective diameters of the sealing surface, or outer dimension, of the distal tip include 9-11 mm, 11-13 mm, 13-15 mm, 15-17 mm, and 17-19 mm. The material comprising the nasal cushions is preferably 20-60 Shore A durometer. Nasal cushions are preferably made of an elastomer such as silicone, urethane or Santropene; or, as another example, can be comprised of a thermoplastic elastomer such as a urethane-PVC blend. As yet another example, nasal cushions can be made of a thermoplastic, such as plasticized PVC, or a styrene material.

FIG. 16C illustrates a front view of an embodiment of a nasal cushion in accordance with an embodiment of the present invention. Nasal cushion 74 may have an angulated axis. Nasal cushion 74 preferably may be angled with an angle h, which preferably facilitates alignment with the nostril foramen. The angle h is preferably 5-20 degrees, more preferably 10-25 degrees. The nasal cushions 74 may be symmetric so that the same cushion can be used for either the left or right cushion, and may comprise an alignment key feature 328, which preferably facilitates correct rotational alignment with a manifold. It is anticipated that, when installed on a manifold, each nasal cushion would be angled inward (toward the midline of the user's face). Optionally, multiple pairs of angled cushions, with each pair having a different angle h, can be provided to the clinician or user to give the user a greater range of fit options.

FIG. 16D illustrates an alternative embodiment of a nasal cushion in accordance with an embodiment of the present invention. In this embodiment, the nasal cushion 73 includes a base with a curve g, rather than a planar surface. Curve g preferably facilitates mating of the base of the nasal cushion with the manifold, and may be preferred, for example, where the superior surface of the manifold is curved. The curve of the base of the cushion may preferably be manufactured to match the curve of the superior surface of the manifold with which it is to be use, thus facilitating mating together of the nasal cushion and manifold and preferably producing a more secure fit with less or no leakage.

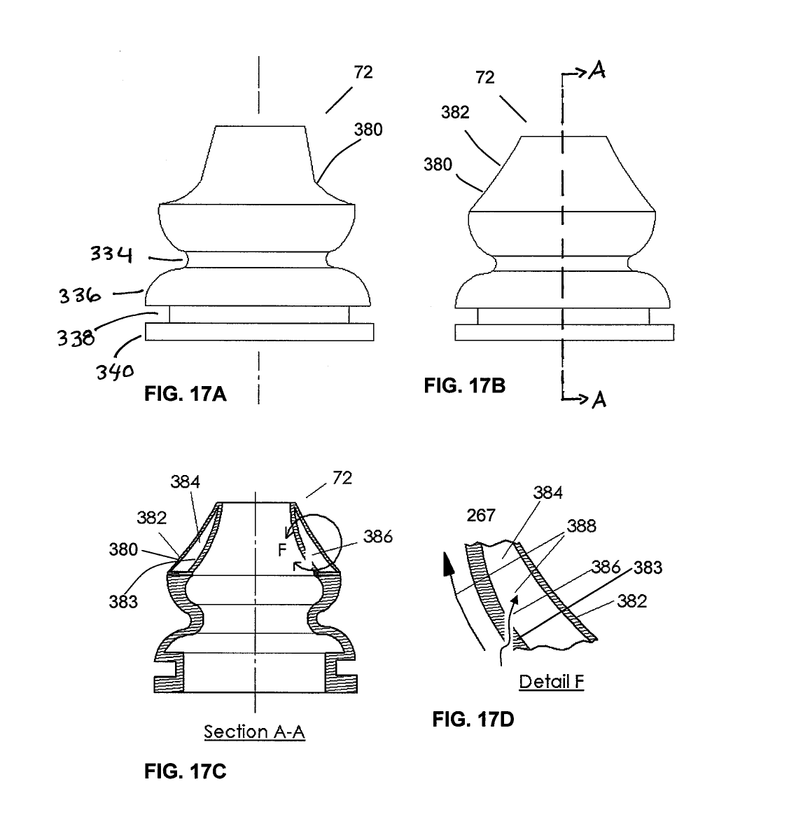

FIGS. 17A-17D describe an alternate embodiment of a nasal cushion in accordance with an embodiment of the present invention. In nasal cushion 72, sealing surface 380, which preferably is capable of sealing against the nostril, is the outer surface of an inflatable or distend-able outer wall 382. FIG. 17A illustrates a front or side view of the cushion 72 with a curved sealing surface 380, which is dimensioned to approximately mate with the nostril opening of the user. In FIG. 17A, the nasal cushion 72 is not inflated or distended. FIG. 17B shows outwardly distended, inflated, nasal cushion 72. FIG. 17C illustrates a cross section of FIG. 17B at line A-A. As can be seen in FIG. 17C, nasal cushion 72 includes outer wall 382 which bears sealing surface 380, inner wall 383, and a pocket 384 between outer wall 382 and inner wall 383. Pocket 384 communicates with the ventilation gas through a port 386 in inner wall 383. Thus, surface 380 of the nasal cushion preferably becomes distended when pocket 384 is inflated by the ventilation gas pressure. FIG. 17D illustrates a detailed view of area F of FIG. 17C. As inspired ventilation gas flow 388 flows through the nasal cushion at a positive pressure, the sealing cushion sealing surface 380 preferably distends by ventilation gas 388 entering the pocket 384 through the port 386. The opening of the port 386 preferably may be angulated to be substantially collinear with the direction of some of the inspired ventilation gas flowing through the nasal cushion, preferably thereby facilitating entry of inspired gas into the pocket 384, inflation of the pocket, and distention of the outer wall 382, and thus preferably effecting a conformal seal around the contacting surfaces of the nostril rim between surface 380 and the nostril rim. Conversely during exhalation, the direction of exhaled flow through the nasal cushion (not shown) is substantially 180 degrees from the port opening, and hence preferably creates a venturi effect at the port opening, which preferably draws out some of the air in the pocket thereby relaxing the outer wall 382 and relaxing the sealing surface 380 so that it is not tensioned against the nostril tissues. This mechanism preferably provides an effective seal during inspiration, when it is needed, by having a slightly higher gas pressure in the pocket, than in the nostrils outside of the nasal cushion, thus minimizing leaking past the seal. Preferably, lower forces are exerted against the nostril during exhalation when sealing is not needed. The pocket 384 can be created by any suitable method, for example, by a two-shot elastomer molding process, in which the cushion is molded with the inner wall in the first shot, and then over-molded with the outer wall in the second shot. As illustrated in FIGS. 17A-17C, nasal cushion 72 also includes other features previously described, such as an inward curve 334 and pillow 336, which preferably provide increased compressibility and flexion ability of the cushion in order to better control, regulate, or limit the amount of force the nasal cushion exerts on the nose and nostrils. The cushion may also include a groove 338 to mate with an attachment rim and opening on a manifold, and a base retaining flange 340, which may be inserted through the manifold gas flow openings to better retain the cushion in place.

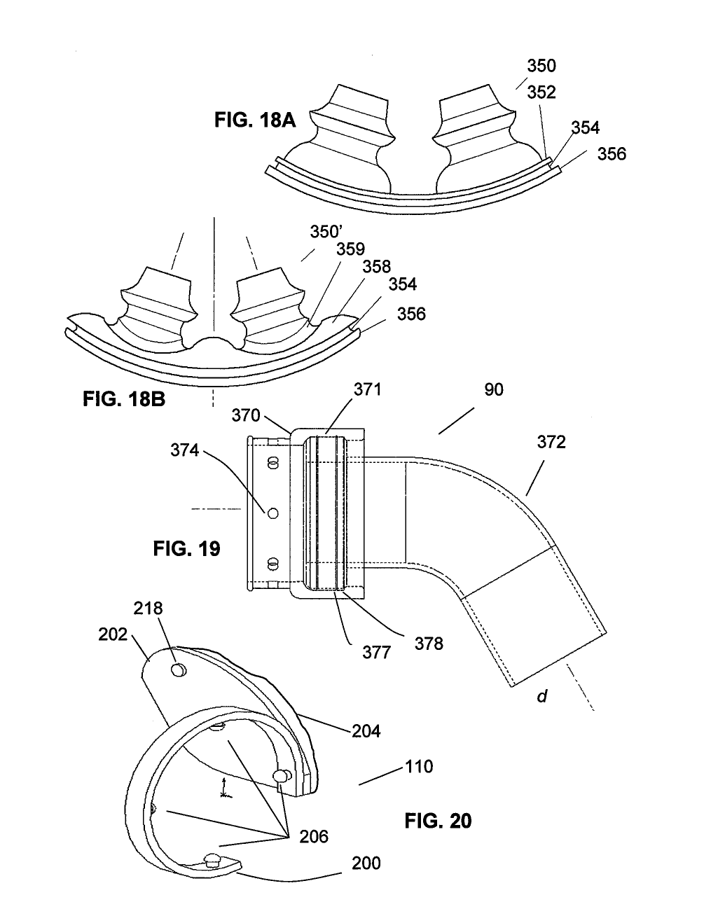

FIG. 18A illustrates an alternative embodiment in accordance with an embodiment of the present invention, in which two nasal cushions are integrated into a one piece nasal cushion assembly 350. In such embodiments, both the left and right nasal cushions are preferably attached to a base that preferably comprises an upper flange 352, a groove 354, and a lower flange 356. The groove preferably mates with an opening on the superior surface of a manifold (not shown), and the upper and lower flanges preferably capture the manifold superior wall around the opening in the manifold. The cushion assembly 350 may be provided in a variety of sizes, shapes, spacings, and angulations. Further, the assembly may be constructed of a material that can be re-formed or reshaped by the caregiver or user in order to mate with the user's anatomy. Reshaping may be made possible by many suitable methods, such as by fabricating the base with a thermoset material that can be heated and reformed, or by including malleable members within the construction of the assembly to bend and flex it in the shape desired. In another embodiment, as shown in FIG. 18B, the nasal cushion assembly 350′ can include a flexible base, to facilitate the nasal cushions or nasal cushion assembly flexing and absorbing forces that the manifold and nasal cushions may encounter during use, preferably better maintaining comfort and sealing integrity. The flexible base may include a compressible base pillow 358 and flexion grooves 359.

FIG. 19 illustrates an embodiment of a swivel elbow connector assembly 90 according to an embodiment of the present invention. Swivel elbow connector assembly 90 may be used to unilaterally connect a manifold to a ventilation gas supply hose. The swivel elbow connector assembly 90 preferably includes at least two pieces, a distal end connector 370, which preferably attaches to a manifold, and an elbow connector 372, which preferably attaches to the distal end connector 370 at its distal end and to a ventilation gas supply hose at its proximal end. Distal end connector 370 and elbow connector 372 preferably connect to each other with a rotatable swivel joint 371. The mating diameter surfaces preferably include (a) a small distance of separation to allow free rotation, for example an approximately 0.005″ gap between the inner diameter of distal end connector 370 and the outer diameter of elbow connector 371, and (b) a raised ridge 378 on the diameter of one of the connectors and a mating groove 377 on the diameter of the other connector, which preferably creates a low gas pressure seal between the two connectors. There may optionally be a small gas flow path between the two connectors to allow leakage of preferably less than 100 ml/min at pressures of 15 cmH₂O. In FIG. 19, the mating diameter surfaces between distal end connector 370 and elbow connector 372 are shown as straight in the cross sectional side view, which preferably facilitates rotational swiveling between the two pieces. However, the mating surfaces can alternatively be curved, for example partial spheres, to allow swiveling of the elbow in multiple planes, for example in the sagittal plane, transverse plane, coronal plane, or any combination thereof. The elbow section of elbow connector 372 is shown with an angle d. Angle d is preferably greater than 90 degrees, which preferably reduces resistance to airflow going through the bend of the connector, and is preferably less than 180 degrees, which preferably bends the gas supply attachment to a desired direction, preferably away from the center of the user's face, away from the cheekbone, and/or away from the ear. Most preferably, angle d is between 100-140 degrees. The distal end of distal end connector 370 preferably includes a straight diameter surface in or on which through holes 374 are located for attachment of manifold connecting ring 200 or 200′ (not shown). Distal end connector 370 is preferably attached to a manifold so that the holes 374 in the connector 370 line up with the holes in a manifold, such as holes 244 as shown in FIG. 5 or holes 278 as shown in FIG. 6. The connector and manifold can include a key way feature (not shown) to facilitate the alignment of the holes. When connected, barbs on the connecting ring 200 or 200′ may press through the two sets of aligned holes in the manifold and distal end connector, to secure the ring, manifold and distal end connector together. Alternatively, the distal end connector can include protruding barbs which would protrude radially outward and press through the holes in the manifold and holes in the connecting ring. The barb and hole style of connection is exemplary, and other forms of connection are included in some embodiments the invention; for example a snap connection, an interference fit connection, or tongue and groove connection may be utilized. Also, optionally, two or more of the ring, connector and manifold parts may be pre-assembled by bonding or joining the parts in manufacturing, and provided to the user pre-assembled or semi-pre-assembled. The proximal end of the elbow connector 372 preferably may be adapted to connect to a gas supply hose, preferably a flex hose that can flex to absorb forces and be positioned where most comfortable or desired. Swivel and elbow connectors are preferably comprised of a thermoplastic, such as polypropylene, polyethylene, polysolfone, nylon or PVC, and preferably have a wall thickness of 0.030-0.060″.

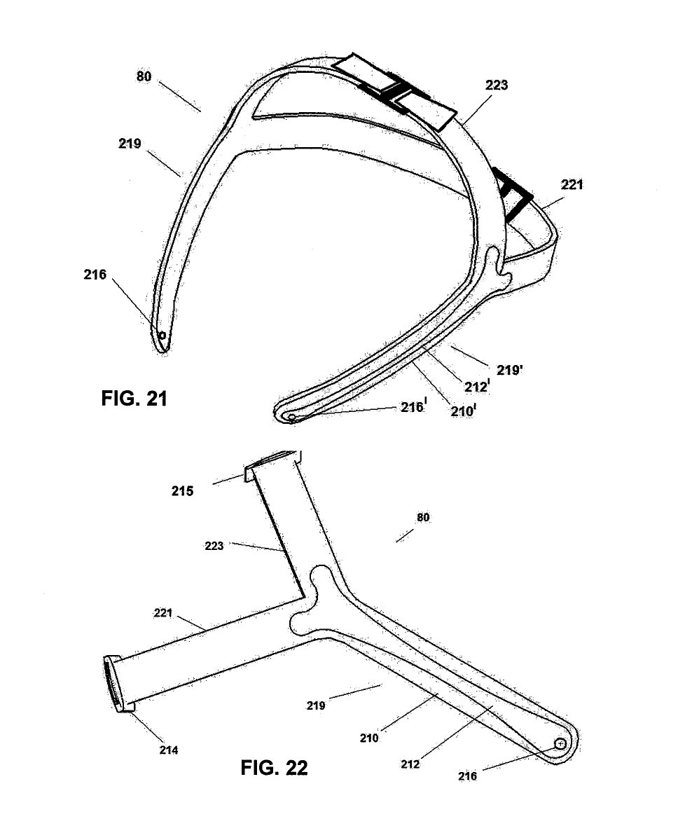

FIG. 20 illustrates in more detail an embodiment of a right interconnecting assembly. An embodiment of a left interconnecting assembly may preferably be a mirror image of the right assembly. The interconnecting assembly 110, which may also be called a manifold-head strap attachment assembly, includes a connecting ring 200 and attachment plate 202. The connecting ring can be a partial ring, preferably greater than 90 degrees in circumference, or can be a complete 360 degree ring, and preferably comprises barbs 206 protruding radially inward, which prefereably may be spaced and dimensioned to be aligned with and pressed through manifold ring attachment holes, such as holes 244 as shown in FIG. 5 or holes 278 as shown in FIG. 6, and distal end connector through holes 374, illustrated, for example, 19. The barbs and holes are exemplary only, and other forms of attachments are included in some embodiments of the invention. Preferably, connecting ring 200 seats into a mating groove on a manifold (such as manifold atttachement ring groove 274 in FIG. 7), preferably creating a flush surface between connecting ring 200 and the manifold. Optionally, the edges of connecting ring 200 can be rounded so that when attached to a manifold, the assembled surface is preferably approximately smooth and preferably free from sharp edges. Connecting ring 200 preferably can be rotated about the manifold, thus varying where it is attached to the manifold in order to better obtain the appropriate angle of nasal cushions in the sagittal plane, facilitating better alignment of nasal cushions with the nostril foramen in the sagittal plane. Head strap attachment plate 202 preferably interconnects connecting ring 200 to a forward strap of a head strap assembly 80. Attachment plate 202 is preferably angled or curved, so that when in use, it preferably is capable of sweeping laterally and posteriorly from its anterior edge to its posterior edge, such that its shape preferably better matches the curvature of the face lateral to the nose, and it preferably maintains better contact with the skin. The anterior side of attachment plate 202 preferably may be semi-rigid, such as Shore 60-90 A hardness, for example, preferably in order to give the plate the structural rigidity sufficient so that the strapping forces from the head strap assembly are effectively transferred to the manifold to stabilize the manifold in proper position under the nose without excessive play or looseness. The posterior or skin side of the attachment plate 202 preferably includes an attachment plate pad 204, which is preferably made of a soft, pliable, resilient, and/or non-abrasive material and preferably cushions attachment plate 202 against the skin, absorbs strapping forces, and/or provides a more comfortable fit. Attachment plate 202 and connecting ring 200 may optionally also include a force absorption section, such as a spring section, which preferably may facilitate absorption of sudden forces and avoidance of shifting of the manifold if is accidentally exposed to sudden or undue forces. Attachment plate 202 preferably includes a connection joint to interconnect the plate and head strap assembly; preferably a swivel joint is used, although other types of joints may be used. In the embodiment shown in FIG. 20, the connection joint is depicted as a protruding barb 218 over or onto which a mating hole on the head strap assembly (such as 216, FIG. 21) may be pressed. Other forms of attachment between the plate and head strap assembly are also included in some embodiments of the invention, such as a snap, or a tongue and groove. In a preferred embodiment, attachment plate 202 lies against the skin, and the forward head strap is placed over the plate; however the opposite arrangement, in which the forward head strap is placed against the skin and attachment plate 202 is placed over the strap, is also included in some embodiments of the invention. In the latter case, if barbed connection, for example, is used, the barb preferably protrudes from forward head strap stiffening member and a hole in attachment plate 202 is snapped over the barb. The plate can optionally be fabricated with a malleable or pliable material, such that the clinician or user can shape the curve into a shape comfortable to the user. Alternatively, the plate can include a malleable strip inside a soft substrate material, for example a 90 Shore A thermoplastic PVC material encased in a 10-30 Shore A elastomer material. The rotational joint formed by the connection between attachment plate 202 and the head strap assembly preferably allows for a rotation between the two assemblies, preferably rotation of up to 90 degrees. The rotational joint can, for example, be free to rotate based on the forces from the prevailing anatomy; or can be resistant to rotation, such that it must be manually rotated to a desired setting; or can include discrete rotational settings, for example in 1-15 degree increments, preferably 3-5 degree increments, in order for the user to be able to set the angle to the most desired angle. In the latter case, the joint can be set at the rotational angle most desirable for the user and preferably will resist rotation once it is set. The settings can be provided, for example, by raised channels on the anterior side of the attachment plate and mating notches in the posterior side of the forward strap stiffening member which is pressed against the attachment plate. The attachment plate is preferably 0.5-1.5″ in anterior-posterior length, 0.25-0.75″ in height, and 0.080-0.160″ in thickness including the plate and, optionally, attachment plate pad. By preferably including two independent adjustments between the manifold and forward head strap—such as one rotational attachment and one pivoting attachment—the attachment assembly preferably contributes to the adjustability and comfortable fit of the overall assembly, including the manifold and nasal cushions, to each individual user.

FIGS. 21 and 22 illustrate an embodiment of a head strap assembly in more detail. FIG. 21 shows an isometric view of the head strap assembly 80, and FIG. 22 provides a side view. The assembly includes a forward left and right head strap 219′, 219 comprising a first material 210′, 210 and a forward left and right stiffening member 212′, 212 sewn or attached to the first material 210′, 210; and a rear strap 221, preferably having a buckle 214; and, optionally, a top strap 223 having a buckle 215. The head strap assembly preferably may be connected to a manifold with using attachment plate assemblies 110, 110′ (such as in FIGS. 2, 3, and 20). The head strap assembly preferably applies tension forces on the manifold in the superior-posterior direction, which creates a sealing force of nasal cushions (such as nasal cushions 70 shown in FIG. 1) against the nostrils. Any undesirably excessive force may preferably be absorbed by one or more of attachment plate pads (such as 204, 204′ in FIGS. 3 and 20), by the manifold, which is preferably comprised of resilient material, by the optional nasal cushion flex and compression feature, by the optional skin pad on the posterior side of the manifold (such as 230 in FIG. 11), or by any combination thereof. In some embodiments of the invention, head straps may be comprised of a fabric or elastomeric material, or a combination thereof. The straps, if elastomeric, can be, for example, a foam, neoprene, silicone, urethane, rubber, or urethane-PVC blend. The strap can optionally include a primary non-elastomeric material that is interspersed with an elastomeric or rubber material in order to give the strap stretch and compression properties, thereby facilitating comfortable fitting of the overall head strap assembly to a variety of anatomies and head sizes. The elongation of the straps preferably may be 50% at 0.2-4.0 lbs tension, and more preferably 1.0-2.0 lbs tension, except in the area of the stiffening members 212, 212′, which preferably resist elongation at these forces. Preferable materials for the stiffening member are materials with a hardness of 60-90 Shore A, and can be for example PVC, polyethylene, nylon, polypropylene, Ultem, or polysolfone. Preferably the strap elastomer material may be 0.040-0.120″ thick, and the stiffening member preferably may be 0.010-0.050″ thick. In some embodiments of the invention, head straps can be fabricated from a medium soft material, such as a material with 10-40 Shore A hardness, and can include a harder stiffening member inside of and surrounded by, or attached to, the material; for example, the straps may include a 0.020 strip of thermoplastic material surrounded by an elastomer. In some embodiments of the invention, stiffening member may be a strip of thin light weight metal, such as, for example, copper, brass, or nickel. Near the anterior end of the forward left and right straps, the head straps and/or stiffening members of the head straps preferably include a mating attachment feature to attach the head straps and/or stiffening members of the head straps to the attachment plates 202, 202′, which preferably attach the head strap assembly to a manifold. In the embodiment shown in FIGS. 21 and 22, the mating attachment feature is shown as a hole 216 through the head strap, including the stiffening member, which connects over the attachment plate barb 218 (such as shown in FIG. 20); however, this mating attachment feature is only exemplary and other attachment means are included in some embodiments of the invention. Optionally, each of stiffening members 212, 212′ can be separated into two or more members on each of the left and right sides and arranged so that the forward straps can elongate in order to further facilitate a more customized fit to the individual user. The top and rear head straps preferably include either buckles or Velcro attachments in order to tension the straps as necessary to better mate with the individual's anatomy. Other forms of attachment, such as snaps, buttons, or clasps, may also be used to fasten the head strap(s). Whatever kind of attachment mechanism is used, it is preferably adjustable. For example, several sets of snaps may be provided. The head strap assembly preferably may be available to the user in more than one size, preferably at least two sizes for adults. The forward straps are preferably 3-6″ in length, the rear strap is preferably 8-15″ in length, and the optional top strap is preferably 8-15″ in length.

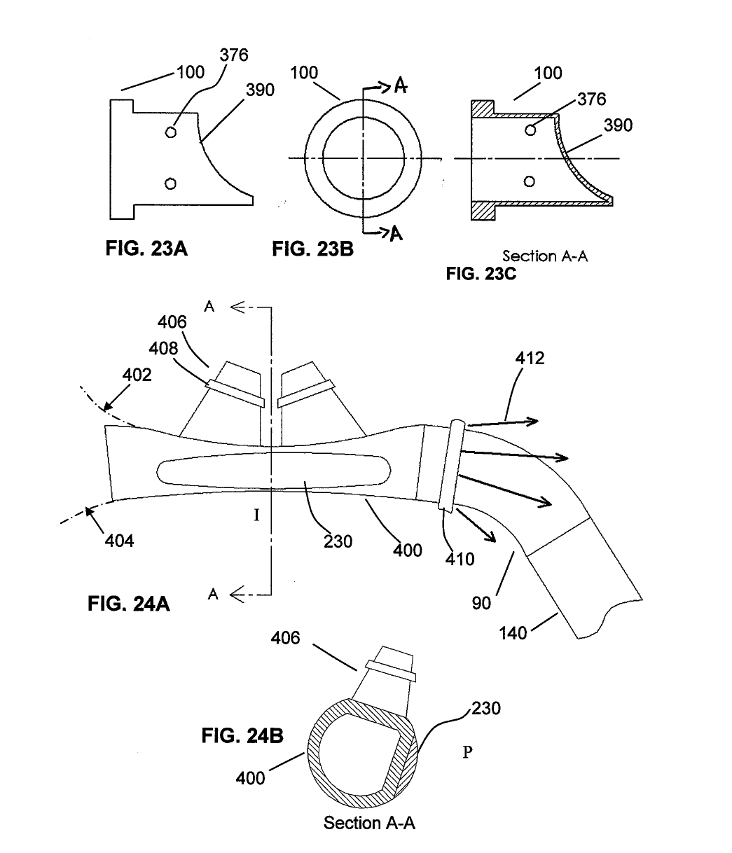

FIGS. 23A to 23C illustrate an embodiment of a manifold end cap, which is preferably used to unilaterally seal one side of the manifold. End cap 100 is preferably configured to be slidably inserted into a lateral end of a manifold. End cap 100 preferably seals, or substantially seals, from gas flow, the lateral end of the manifold into which it is inserted. FIG. 23A illustrates a side view of the cap 100 showing connecting ring holes 376 and a ramp 390. FIG. 23C illustrates a cross sectional view through the end view described in FIG. 23B, showing attachment ring holes 376 and ramp 390. Ramp 390 preferably facilitates laminar air flow in and out of the manifold and nasal cushions by reducing or eliminating turbulent airflow mixing at the end of the manifold that could be caused by the closed end cap. Ramp 390 is optional, and end caps according to some embodiments of the present invention may include other internal profiles instead of a ramp, such as, for example, flat, concave, convex, or multiply curved or arcuate surfaces. Attachment ring holes 376 preferably match with connecting ring attachment holes in a manifold (such as holes 244 as shown in FIG. 5 or holes 278 as shown in FIG. 6), and barbs, such as barbs 206 on connecting ring 200 or 200′, are preferably pressed therethrough, thereby connecting a manifold, end cap, and head strap interconnecting assembly. Alternatively, the end cap can include protruding barbs which would protrude radially outward and press through the holes in the manifold and holes in the connecting ring. The end cap may be made of materials that are similar to those of the distal end connector.

In an optional embodiment, the left and right attachment plate 202′, 202 and manifold posterior skin pad 230 are combined in a continuous assembly. In such embodiments, the combined assembly is longer than the width of the nose, and the head strap assembly forward straps are attached to the left and right sides of the assembly. The assembly can be fixed to the manifold, or can be removably attached to the manifold.

FIG. 24A illustrates an alternate embodiment of the present invention, in which the nasal ventilation interface includes a manifold 400 which possesses a concave or superior sweeping curve 402 on the superior or top side and a concave or inferior sweeping curve 404 on the inferior or bottom side of the manifold. The radius of the curves, as well as the length of the manifold, can be varied to provide varied sizes to patients with varying size faces. For example, a clinician can fit a patient with an appropriately sized and curved manifold, or multiple manifolds can be provided to a patient. The concave curve on the superior surface preferably allows the manifold to better conform to the geometry of the face under the nose and to the sides of the nose, with minimal obtrusiveness. For example, a non-curved manifold may require more space between the nasal cushion base and the nostril, thus making the manifold larger. The opposing concave curve on the bottom surface of the manifold preferably allows the manifold to better conform to the anatomy of face above the upper lip. The curve preferably follows the curvature of the lip, and hence creates a shape that preferably does not interfere with the lip. For example, a non-curved manifold could impinge on or contact the upper lip in the center of the lip, because the top to bottom width of the manifold preferably is wide enough to accept the connector for the ventilation gas supply. The narrowing of the manifold top to bottom width in the central section of the manifold, by using the opposing concave curves on the superior and inferior surface, and the manifold's widening at the lateral ends, create a design that, for some patients, may facilitate avoidance of contact with the lips, preferably be large enough to connect to the ventilation gas supply, and preferably optimize fit with the nose, thus resulting in a best possible fit and least possible obtrusiveness for certain patients. It might be thought that the restriction of the cross-sectional dimension near the center of the manifold could unnecessarily increase flow resistance; however, advantageously in this design, because 50% of the flow does not need to cross the restricted section because it enters the proximal nasal cushion, and only 50% needs to cross the restriction, there preferably is no measurable increase in overall flow resistance of the design. Also shown, the concave curve on the top surface preferably positions the nasal cushions at an inward tilt, which preferably better conforms to the anatomy of the angles of the nostril.

In addition to the opposing concave curves feature and function described above, FIG. 24A illustrates additional detail of an optional skin pad on the posterior side of the manifold. In this embodiment, the skin pad 230 may be used to tilt set the angle of the manifold in the sagittal plane, so that nasal cushions 406 may be better angularly aligned with the angle of the nostrils in the sagittal plane. Further skin pad 230 preferably may be designed to absorb forces applied by the head strap assembly securing straps which pull the manifold posteriorly against the user's skin. The skin pad can be integrally formed with the manifold, or can be a separate piece attached to the manifold. It the latter case the skin pad can be removeably attachable to the manifold so that different sizes can be attached to customize and individualize the fit to the user, and to replace and clean the pad when needed. The skin pad preferably covers a wide section of the manifold, which preferably helps to stabilize the angle of the manifold and to absorb or distribute the strapping forces. The skin pad may optionally extend beyond the lateral ends of the manifold and may optionally be used for connection of the manifold assembly to the head strap assembly, in which case the skin pad lateral ends will include a rigid insert with an attachment feature to attach the forward straps of the head strap assembly. The attachment feature is for example a barb or hole as described previously. The skin pad preferably may be comprised of a viscoelastic or highly compliant material. FIG. 24B illustrates a cross section of the manifold of FIG. 24A at line A-A and illustrates the skin pad attached to the posterior side of the manifold.

Also shown in the embodiment of FIG. 24A, optional exhaust vent channels may be placed in a connector 410 of the gas supply assembly swivel elbow connection 90 in order to allow for exhalation air flow 412. As another alternative, optional exhaust vent channels may be placed in the gas supply assembly swivel elbow connection 90 in order to allow for exhalation air flow. The vents may preferably be biased so that they are less resistive to flow in the exhaled direction and more resistive to flow in the inhaled direction. Preferably, the openings of the channels inside the assembly are positioned on a surface that is struck by airflow only in the exhaled direction. As another alternative, a portion of the connector 410 can move within the elbow swivel connector assembly 90 with the inertia of the gas flow when gas is moving in the exhaled direction such that the movement exposes the vent channels, such that the vents are more closed during inspiration and less closed during exhalation. The surfaces of the moving section of the connector or the opposing surfaces preferably may be comprised of soft material to prevent noise related to the moving parts. The embodiment of FIG. 24A also shows nasal cushions 406 attached to the manifold wherein the manifold tilts the cushions inward, wherein the inward edge is straight and the outward edge is tilted inward, so that the inward and outward edges mate with the typical anatomy of the nostril wherein the nostril septum is substantially straight and the nostril outer wall is substantially angled inward. An optional sealing step 408 on the nasal cushions may also be tilted to better mate with the nostril opening, which is usually tilted. The nasal cushions preferably may be tapered from the proximal base to the distal tip. The features shown in the embodiment of FIG. 24A may be used individually or in any combination, and can be used in combination with features described in other embodiments of the invention, for example the posterior curve of the manifold, or the detachable nasal cushions.

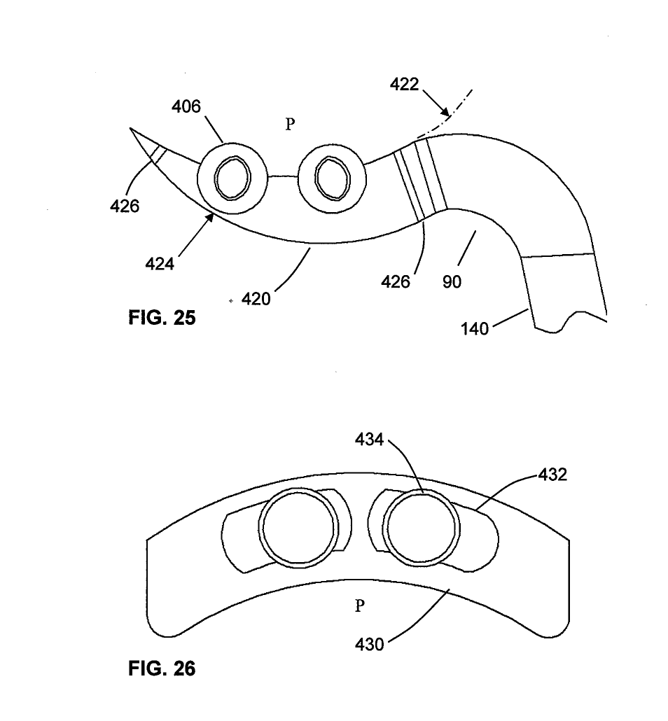

FIG. 25 illustrates an embodiment of the present invention in which a manifold 420 has a sweeping, curved and tapered shape. The posterior or patient skin side P of manifold 420 preferably comprises a symmetrical concave curve 422, which preferably matches the contours of the face under the nose. The anterior side of the manifold preferably comprises a non-symmetrical curve 424 to taper the width of the manifold from right to left. The end of the manifold with the reduced dimension caused by the taper is placed on the side of the face on which the user is sleeping, and thus preferably reduces the bulkiness of the interface in that area, preferably making it more comfortable to sleep on that side. The gas supply assembly may be attached to the manifold on the lateral side opposite the tapered side. Both ends of the manifold have a head strap connector mating feature 426, such as a groove, to attach the manifold to a head strap assembly, which secures the manifold to the face. This embodiment preferably is made available to the user in version with the taper on the left and a version with the taper on the right version, so the user has an option of which side to sleep on. Both versions can be provided to the user in a kit so that the user can switch between versions as desired.

FIG. 26 illustrates an embodiment of a manifold in which the spacing between the nasal cushions can be adjusted. The manifold 430 includes a self sealing adjustment slot 432 in which nasal sealing cushions 434 are slide-able.

In another optional embodiment, the manifold, or the nasal cushions, can include pores which can receive an aroma therapy substance. The pores are configured for slow release of the substance, so that it is depleted from the pores in the course of 1-8 hours. Optionally, the interface assembly can include a receptacle section which receives a cartridge of aroma therapy. In addition, the nasal interface assembly includes a connection for connecting a supplemental supply of oxygen rich gas, and a connection for actively removing CO₂ rich gas from the conduits of the assembly.

In an optional embodiment, thin wall rigid cylindrical sleeves are placed inside the manifold ends to create a rigid lateral end of the manifold. The end cap or swivel elbow connector are then pressed into the cylindrical sleeves.

It can be appreciated that the nasal interface device can comprise any, some or all of the described embodiments and that the described embodiments can be combined in ways not explicitly described. Also, while most of the embodiments described relate to long term or repeated use of the device, such as with OSA, it can be appreciated that there are non-OSA ventilation uses that would also benefit from these embodiments, such as PAP therapy for COPD, anesthesia delivery and recovery, mechanical ventilator weaning, NIV, outpatient surgery use, and emergency ventilation. Further, it should be appreciated that in addition to CPAP or VPAP ventilation, embodiments of the invention can be used for other forms of mechanical ventilation such as CMV, SIMV, etc. For some embodiments, in some applications of the device, it is beneficial for the gas supply to be connected to both the left and right lateral ends of the manifold. Finally it should be appreciated that with the necessary modifications, the device can be reusable or disposable and can be adapted for adult, pediatric or neonatal use. While the invention has been described in detail with reference to the preferred embodiments thereof, it will be apparent to one skilled in the art that various changes and modifications can be made and equivalents employed, without departing from the present invention.

Although a ventilator is not explicitly illustrated or described with relation to all embodiments described herein, the embodiments of the present invention are preferably used in conjunction with a ventilator, non-limiting examples of which include CPAP, VPAP, and auto-titrate PAP ventilators, as well as mechanical ventilators, jet ventilators and other unconventional ventilators.

Although the foregoing description is directed to the preferred embodiments of the invention, it is noted that other variations and modifications will be apparent to those skilled in the art, and may be made without departing from the spirit of scope of the invention. Moreover, features described in connection with one embodiment of the invention may be used in conjunction with other embodiments, even if not explicitly stated in the foregoing.

The present invention may be embodied in other specific forms without departing from its spirit. Such additional embodiments and forms will be clear to one skilled in the art provided with the disclosure herein. The described embodiments are to be considered in all respects only as illustrative and not restrictive.

1. A nasal interface assembly for providing ventilation gas, comprising:
(a) a tubular manifold adapted to be positioned inferiorly to the nostrils and superior to the mouth comprising: (1) an axially tubular structure comprising compound arcuate curves curving bilaterally from a manifold midline in a lateral and posterior direction, (2) a left lateral end and a right lateral end, (3) at least two gas flow openings on the superior side of the manifold, and (4) and a closure connected to the one lateral end;
(b) a pair of tubular nasal cushions each comprising a proximal end base attached to one of the at least two gas flow openings, and each extending to a distal end adapted to impinge with and seal against the nostrils;
(c) a ventilation gas supply assembly comprising a distal end adapted to connect to the lateral end of the manifold opposite the lateral end connected to the closure, the connection adapted to rotate in at least one plane, and a proximal end adapted to attach to a ventilator;
(d) a head strap assembly; and
(e) a connector connecting the head strap assembly to the manifold, the connector comprising two movable connections, wherein a first movable connection between the manifold and connector allows rotation of the manifold cross section in the sagittal plane, and a second movable connection between the head strap and connector allows pivoting of the head strap in the inferior-posterior direction.

2. The nasal interface assembly of claim 1, wherein the compound arcuate curves of the manifold are shaped to match the anatomy of a user's face between the nose and mouth and to the sides of the nose and wherein the curves are stabilized to maintain substantial contact of the skin side of the manifold with the user's anatomy.

3. The nasal interface assembly of claim 1, wherein the compound arcuate curves of the manifold include a concave curve on the superior side of the manifold in the coronal plane curving or angling superiorly from the midline, wherein the concave curve comprises a substantially V-shaped or U-shaped curve symmetrical about the midline, and wherein the substantially V-shaped or U-shaped curve comprises a 110-170 degree included angle.

4. The nasal interface assembly of claim 1, wherein the compound arcuate curves of the manifold include a concave curve on the superior side of the manifold in the coronal plane curving or angling superiorly from the midline and a concave curve on the inferior side of the manifold in the coronal plane curving or angling inferiorly from the midline.

5. The nasal interface assembly of claim 1, wherein the compound arcuate curves of the manifold further comprise a taper wherein the taper transitions from a first larger cross sectional dimension at a medial location to a second smaller cross sectional dimension at a lateral location.

6. The nasal interface assembly of claim 1, wherein the manifold further comprises at least one exhalation flow vent port in a location selected from the group consisting of: at least one channel in the wall of the manifold at a inferior-anterior location opposite to and substantially aligned with the gas flow openings, at least one channel in the manifold lateral end closure, at least one channel in the ventilation gas supply assembly substantially aligned with the direction of exhaled gas flow.

7. The nasal interface assembly of claim 1, wherein the cross sectional profile of at least one section of the manifold is shorter in a first axis and longer in a second axis orthogonal to the first axis, wherein the second axis is positioned parallel to the face.

8. The nasal interface assembly of claim 1, wherein the manifold further comprises a middle section in the location of the gas flow openings, and wherein the middle section comprises (i) a cross sectional superior surface in the sagittal plane that angles inferiorly 5-30 degrees from the anterior side to the posterior side and (ii) a cross sectional posterior surface in the sagittal plane that angles posteriorly 5-30 degrees from the superior side to the inferior side.

9. The nasal interface assembly of claim 1, wherein the manifold comprises a material having Shore 10-30A hardness, and further comprises at least one stiffening member, wherein the stiffening member is selected from the group consisting of: a strip of semi-rigid plastic, a strip of metal alloy, a radial rib, and an axial rib.

10. The nasal interface assembly of claim 1, wherein the manifold comprises a material having Shore 10-30A hardness, and further comprises at least one stiffening member, wherein the stiffening member stabilizes and reduces the compressibility of the tubular manifold structure.

11. The nasal interface assembly of claim 1, wherein the manifold comprises a material having a Shore 10-30A hardness, and further comprises at least one malleable member adapted to allow a user to bend and slightly reshape the curvature of the manifold to fit the user's individual anatomy.

12. The nasal interface assembly of claim 1, wherein the superior surface of the manifold comprises a convolution in the wall around the gas flow openings.

13. The nasal interface assembly of claim 1, further comprising a rigid cylindrical sleeve inside the manifold at each lateral end.

14. The nasal interface assembly of claim 1, wherein the manifold further comprises a groove in the wall near each of the two lateral ends, wherein groove is adapted for attachment of the head strap assembly connector.

15. The nasal interface assembly of claim 1, wherein the manifold further comprises a spacing adjuster adapted to adjust the distance between the nasal sealing cushions, wherein the spacing adjuster is selected from the group consisting of: a self sealing slot, an adjustable ring, and a replaceable adaptor.

16. The nasal interface assembly of claim 1, wherein the manifold comprises separate left and right tubular sections, wherein the two tubular sections are connected by a tubular interconnecting member.

17. The nasal interface assembly of claim 1, wherein the nasal cushions further comprise a round proximal base and an oval distal end and wherein the nasal cushion proximal base and manifold superior side gas flow openings comprise a mating rotatable connection adapted to rotate the nasal cushion about its attachment to the manifold, and further wherein the nasal sealing cushions further comprise: (1) a convolution in the wall near the proximal end base, wherein the convolution is adapted to enable angular flexing or axial compression of the nasal cushion, (2) construction using a material having a durometer of 10 to 50 Shore A, the material selected from the category of: a thermoplastic, an elastomer, or a thermoplastic elastomer, (3) an enlarged effective diameter at a distance 2-10 mm from proximal to its distal end wherein the enlarged effective diameter creates the general configuration of a step, and wherein the enlarged effective diameter is a dimension larger than the nostril rim inner diameter, and is in the range of 7 mm to 20 mm and wherein the step engages the nostril rim to effect a seal and to prevent over penetration of the nasal sealing cushion into the nostril, and (4) a second seal at the distal tip of the cushion which is adapted to seal inside the nostril, the distal tip seal comprising a feature selected from the group consisting of a flare, a ring, an effective diameter larger than the nostril dimension, and an inflatable wall.

18. The nasal interface assembly of claim 1, wherein the manifold superior side comprises a raised tubular extension for attachment of the nasal cushions, and wherein the nasal cushions (a) are adapted to slip over the raised tubular extension, and (b) are comprised of a viscoelastic shape memory material selected from the group consisting of: an elastomer, a hydrogel, and a foam, wherein the material possess a recovery of greater than 80% in 3 seconds from 50% compression.

19. The nasal interface assembly of claim 1, wherein the nasal cushions further comprise an inflatable outer wall sealing surface adapted to inflate by the flow of ventilation gas flowing to the patient, wherein the inflation outwardly expands the nasal cushion outer wall sealing surface to press against the nostril foramen.

20. The nasal interface assembly of claim 1, further comprising a pad attached to the posterior side of manifold, wherein the pad extends to the lateral sides of the nose, and wherein the pad further comprises a connector at the lateral ends to connect to the head strap assembly.

21. The nasal interface assembly of claim 1, wherein the connector connecting the head strap assembly to the manifold further comprises: (1) a left and right connector adapted to be removably and rotatably attachable to the manifold, with at least two rotational attachment positions, (2) a left and right connecting plate connected to the left and right connector at the posterior side of the manifold wherein the plates comprise (a) a pad on the posterior skin side of the plate and (b) a connection joint for connecting to the head strap assembly, and further wherein the head strap assembly further comprises a forward left and right strap comprising: a soft material on the skin size of the strap, and a semi-rigid strip, wherein the semi-rigid strip comprises an attachment means to attach to the attachment plate connection joint, and wherein the attachment means comprises a pivoting connection between the forward strap and the attachment plate adapted to provide multiple rotational positions of the strap relative to the plate.

22. The nasal interface assembly of claim 1, wherein the head strap assembly further comprises a malleable member adapted to shape the assembly and resist deformation of a desired shape.

23. The nasal interface assembly of claim 1, wherein the distal end of the ventilation gas supply assembly further comprises: an elbow connector assembly adapted to connect to one side of the manifold wherein the elbow connector assembly comprises at least one rotational connector adapted to rotate in at least two planes, wherein the rotation allows positioning of the gas supply hose to multiple positions.

24. The nasal interface assembly of claim 1, wherein the manifold and ventilation gas supply assembly are further adapted to removably and switch-ably attach the ventilation gas supply to either the left or right lateral end of the manifold.

25. The nasal interface assembly of claim 1, wherein the axially tubular structure further comprises an axial arc length longer than the width of the base of the nose of the user.

26. The nasal interface assembly of claim 1, wherein the distal ends of the nasal cushions have an outer dimension of 7 mm to 17 mm.

27. A kit comprising a nasal interface assembly comprising:
(a) a tubular manifold adapted to be positioned inferiorly to the nostrils and superior to the mouth comprising: (1) an axially tubular structure comprising compound arcuate curves curving bilaterally from a manifold midline in a lateral and posterior direction, (2) a left lateral end and a right lateral end, (3) at least two gas flow openings on the superior side of the manifold, and (4) and a closure connected to the one lateral end;
(b) a pair of tubular nasal cushions each comprising a proximal end base attached to one of the at least two gas flow openings, and each extending to a distal end adapted to impinge with and seal against the nostrils;
(c) a ventilation gas supply assembly comprising a distal end adapted to connect to the lateral end of the manifold opposite the lateral end connected to the closure, the connection adapted to rotate in at least one plane, and a proximal end adapted to attach to a ventilator;
(d) a head strap assembly; and
(e) a connector connecting the head strap assembly to the manifold, the connector comprising two movable connections, wherein a first movable connection between the manifold and connector allows rotation of the manifold cross section in the sagittal plane, and a second movable connection between the head strap and connector allows pivoting of the head strap in the inferior-posterior direction.

28. A kit according to claim 25, further comprising (a) at least one manifold of a first size and a second manifold of a second size, (b) at least one head strap assembly of a first size and a second head strap assembly of a second size, and (c) at least one pair of nasal sealing cushions of a first size and a second pair of nasal sealing cushions of a second size.

29. A kit according to claim 25, wherein the distal ends of the nasal cushions have an outer dimension of 7 mm to 17 mm.

30. A method for supplying ventilation gas to a person to assist in inflating the lung of the person using a nasal interface assembly, the method comprising:
(a) placing a compound arcuately curved tubular manifold between the user's nose and mouth and stabilizing it against the skin;
(b) removably attaching a pair of tubular nasal cushions to the superior side of the manifold, wherein the nasal cushions to impinge on the nostril rims, and limiting penetration of the cushions into the nostrils by including an enlarged step on each cushion;
(c) connecting a distal end of a ventilation gas supply assembly to a first lateral end of the manifold to create a rotatable connection in at least one plane, and connecting the proximal end of the ventilation gas supply hose to a ventilator;
(d) sealing the second lateral end of the manifold opposite the connection of the ventilation gas supply hose assembly; and
(e) fastening the manifold to the user's face by attaching a head strap assembly to the manifold with at least one adjustable attachment between the manifold and head strap assembly.

31. The method of claim 30 further comprising: switching the ventilation gas supply assembly attachment to the second lateral end of the manifold and switching the sealing of the second lateral end of the manifold to the first lateral end of the manifold.

32. The method of claim 30 further comprising: moving the position of the ventilation gas supply hose from one position on the face to a second position on the face using rotatable connections.

33. The method of claim 30 further comprising securing the user interface to the patient's face by pulling the manifold in a posterior and superior direction using the head strap assembly coupled to the lateral ends of the manifold and extending straps of the head strap assembly to the back and top of the head, and wherein the manifold is rotationally adjusted by the coupling, and the straps are pivoted by the coupling.

34. The method of claim 30 further comprising inflating the nasal sealing cushions with gas supplied from the ventilation gas supply.

35. The method of claim 30, further comprising adjusting the nasal cushions, the adjustment selected from the group of: adjusting the included angle between the nasal sealing cushions, adjusting the space between the nasal sealing cushions, rotating the nasal cushions at the connection between the nasal cushions and the manifold.

36. The method of claim 30, further comprising exhausting exhalation flow, the exhausting selected from the group consisting of: exhausting through ports positioned in wall of the manifold opposite to the nasal cushions, exhausting through the lateral end closure, exhausting through ports in the ventilation gas supply.

37. The method of claim 30 further comprising delivering a supplemental flow of oxygen gas to the patient by connecting a supply of oxygen gas to the manifold.

38. A nasal interface assembly comprising:
(a) a tubular manifold adapted to be positioned inferiorly to the nostrils and superior to the mouth, comprising: (1) an axial length longer than the base of the nose and a left and right lateral end, (2) a centerline arc axis comprising compound arcuate curves with a first section curving bilaterally from the midline comprising a posterior and superior curve, two second sections attached to the left and right lateral end of the first section and curving laterally and posteriorly to the left and right lateral ends of the manifold, (3) at least two gas flow openings on the superior surface of the manifold, (4) a connector on each of the left and right lateral ends of the manifold adapted to attach a gas supply assembly to one end and a closure to the other end, (5) at least one connector on the superior surface adapted to attach two nasal sealing cushions to communicate with the gas flow openings, (6) a connector on each of the left and right lateral ends for attaching a head strap assembly, (7) exhalation exhaust ports;
(b) a pair of tubular nasal cushions comprising a proximal end base adapted to removably attach to a connector on the superior side of the manifold and to be in communication with the gas flow openings, and comprising a distal end adapted to impinge with and seal against the nostrils;
(c) a ventilation gas supply assembly comprising: (1) a distal end elbow connector assembly adapted to attach to one lateral side of the manifold and adapted to rotate in multiple planes, (2) a flex hose in communication with the distal elbow assembly, and (3) a proximal end connection adapted to attach to a ventilator;
(d) a closure adapted to attach to a lateral side of the manifold; and
(e) a head strap assembly comprising: (1) a left and right connector adapted to rotatably attach to the attachment means on the manifold, (2) a left and right attachment plate attached to the left and right connector wherein the left and right attachment plates are adapted with curves to stabilize the plates in contact with the skin, (3) a left and right strap movably connected to the left and right attachment plate and extending posteriorly to above the ears and joining at the rear of the head.
